(19) United States
(12) Patent Application Publication
(10) Pub. No.: US 2023/0136594 A1
(43) Pub. Date: May 4, 2023

(54) TRICYCLIC P2-LIGAND CONTAINING POTENT HIV-PROTEASE INHIBITORS

(71) Applicant: Purdue Research Foundation, West Lafayette, IN (US)

(72) Inventors: Hiroaki Mitsuya, Kumamoto (JP); Arun K. Ghosh, West Lafayette, IN (US); Satish Kovela, West Lafayette, IN (US)

(21) Appl. No.: 17/906,604

(22) PCT Filed: Jan. 18, 2021
(86) PCT No.: PCT/US2021/013821

§ 371 Date: Sep. 16, 2022

Related U.S. Application Data
(60) Provisional application No. 62/991,391, filed on Mar. 18, 2020.

(51) Int. Cl. C07D 493/08

(52) U.S. Cl. CPC C07D 493/08

(57) ABSTRACT

Compounds of formula (I), pharmaceutical compositions comprising compounds of the formula (I), and methods of treating an HIV infection comprising administering an effective amount of one or more compounds of formula (I), or a pharmaceutical composition comprising same.

### CROSS REFERENCE TO RELATED APPLICATION

This application claims priority to U.S. provisional patent application No. 62/991,391, which was filed on Mar. 18, 2020, and which is hereby incorporated by reference in its entirety.

### STATEMENT OF GOVERNMENT SUPPORT

This invention was made with government support under grant A1150466 awarded by the National Institutes of Health. The government has certain rights in the invention.

### BACKGROUND

The AIDS epidemic is one of the most challenging problems in medicine in the 21st century. Among many strategies to combat this disease, highly active antiretroviral therapy (HAART) with HIV protease inhibitors (PIs) in combination with reverse transcriptase inhibitors (RTIs) continues to be the first line of treatment for control of HIV infection. Although such combination therapy has improved quality of life, enhanced HIV management, and halted the progression of the disease, there remain many challenges to treating this devastating disease, including decreasing both the toxicity and complexity of these treatment regimens. In addition, there is a growing population of patients that is developing multi-drug resistant strains of HIV. And there is ample evidence that these strains can be further transmitted.

Even though HAART has had a major impact on the AIDS epidemic in industrially advanced nations, it has not achieved the eradication of human immunodeficiency virus type 1 (HIV 1), in part due to the viral reservoirs remaining in blood and infected tissues. The limitation of antiviral therapy of AIDS is also exacerbated by complicated regimens, the development of drug-resistant HIV-1 variants, and a number of inherent adverse effects. Further, efforts to bring about the optimal benefits of HAART have met with a number of challenges, including (i) drug-related toxicities; (ii) partial restoration of immunologic functions once individuals develop AIDS; (iii) development of various cancers as a consequence of survival prolongation; (iv) flame-up of inflammation in individuals receiving HAART or immune re-construction syndrome (IRS); and (v) increased cost of antiviral therapy. Such limitations of HAART are exacerbated by the development of drug-resistant HIV-1 variants.

There is presently a paucity of antiretroviral drugs or agents that are not only substantially specific for HIV-1, but also devoid of toxicity or side effects in the therapy of AIDS.

### SUMMARY

The disclosure is directed to a compound of the formula (I):

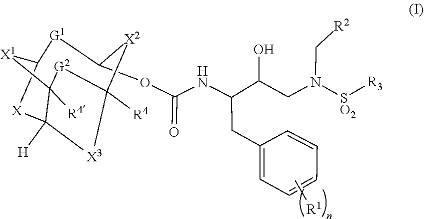

or a pharmaceutically acceptable salt, polymorph, prodrug, solvate or clathrate thereof, wherein:

n is an integer from 0 to 3;

G¹ and G² are each, independently, (—CHR⁵—)ₚ,

p is 0 or 1;

X is (—CHR⁵—)ₘO—;

m is 0, 1 or 2;

X³ is (—CHR⁵—)_{d}O—;

d is 1 or 2;

X¹ and X² are each, independently, (—CHR⁵—)ₘ;

each R¹ is independently alkyl, alkoxy, aryl, heterocyclyl, halo, hydroxy or amino;

R² is alkyl;

R³ is aryl, benzthiazole, benzoxazole, benzofuranyl or indolyl;

R⁴ and R^{4′} are each, independently, H or alkyl; and

each R⁵ is independently H or alkyl.

The disclosure also relates to a pharmaceutical composition comprising a compound of formula (I).

The disclosure relates to a method of treating an HIV infection comprising administering a therapeutically effective amount of one or more compounds of formula (I) to a patient in need thereof.

The disclosure also relates to compounds of the formula (II), which can serve as, among other things, building blocks for the various compounds described herein:

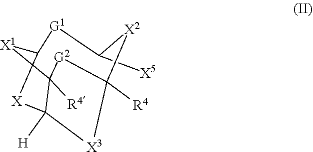

or a pharmaceutically acceptable salt, polymorph, prodrug, solvate or clathrate thereof, wherein the groups X, X¹, X², X³, G¹, G², R⁴ and R^{4′} are as defined herein; and wherein X⁵ is selected from the group consisting of hydroxy, alkoxy, amino, C(O)R, C(O)OR, OC(O)OR, C(O)N(R)₂, OC(O)N(R)₂, C(S)N(R)₂, (CH₂)₀₋₂O(R)C(O)R, (CH₂)₀₋₂N(R)C(O)R, (CH₂)₀₋₂O(R)C(O)OR, (CH₂)₀₋₂O(R)C(O)OR or (CH₂)₀₋₂N(R)N(R)₂, wherein each R can be, independently, hydrogen, alkyl, acyl, cycloalkyl, aryl, aralkyl, heterocyclyl, heteroaryl, or heteroarylalkyl, wherein any alkyl, acyl, cycloalkyl, aryl, aralkyl, heterocyclyl, heteroaryl, or heteroarylalkyl or two R groups bonded to a nitrogen atom or to adjacent nitrogen atoms can together with the nitrogen atom or atoms form a heterocyclyl.

### DESCRIPTION

While the concepts of the present disclosure are illustrated and described in detail in the figures and descriptions herein, results in the figures and their description are to be considered as examples and not restrictive in character; it being understood that only the illustrative embodiments are shown and described and that all changes and modifications that come within the spirit of the disclosure are desired to be protected.

### Compounds

The disclosure is directed to a compound of the formula (I):

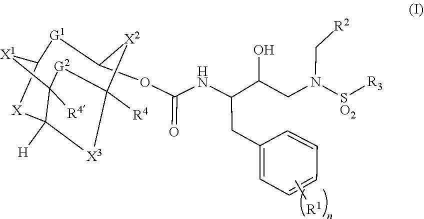

or a pharmaceutically acceptable salt, polymorph, prodrug, solvate or clathrate thereof, wherein: n is an integer from 0 to 3; G¹ and G² are each independently (—CHR⁵—)ₚ, wherein p is 0 or 1 and each R⁵ is independently H or alkyl; X is (—CHR¹—)ₘO—, wherein m is 0, 1 or 2 and each R⁵ is independently H or alkyl; X³ is (—CHR⁵—)_{d}O—, wherein d is 1 or 2 and each R⁵ is independently H or alkyl; X¹ and X² are each, independently, (—CHR⁵—)ₘ, wherein m is 0, 1 or 2 and each R⁵ is independently H or alkyl; each R¹ is independently alkyl, alkoxy, aryl, heterocyclyl, halo, hydroxy or amino; R² is alkyl; R³ is aryl, benzthiazole, benzoxazole, benzofuranyl or indolyl; and R⁴ and R^{4′} are each, independently, H or alkyl. In some examples, n is 0. In addition, (i) X¹ can be (—CHR⁵—)ₘ, with m being 2, and X² being (—CHR⁵—)ₘ, with m being 1, and each R⁵ being the same or different and as defined herein; (ii) X¹ and X² can be (—CHR⁵—)ₘ, with each m being 1, and each R⁵ being the same or different and as defined herein; (iii) X¹ can be (—CHR⁵—)ₘ, with m being 0, X² being (—CHR⁵—)ₘ, with m being 1, and each R⁵ being the same or different and as defined herein; or (iv) X¹ and X² can be (—CHR⁵—)ₘ, with m being 0, provided that at least one of G¹ and G² is (—CHR⁵—)ₚ, wherein at least one p is 1 and each R⁵ is independently H or alkyl.

In any instance of (i), (ii), (iii) or (iv) X can be O, X³ can be O or X and X³ can be O. In addition, in any instance of (i), (ii), (iii) or (iv) at least one p is 0, such that at least one of G¹ and G² is a bond. In any of the foregoing examples, R⁴ and R^{4′} can each, independently, be H or alkyl. For example, R⁴ can be H. In another example, R^{4′} can be H. In yet another example, R⁴ and R^{4′} are each H. In another example, each p is 0; X and X³ are each O, X¹ and X² are each, independently, (—CHR⁵—)ₘ; and R⁴ and R^{4′} are each H. In still another example, each p is 1; X and X³ are each O, X¹ and X² are each, independently, (—CHR⁵—)ₘ; and R⁴ and R^{4′} are each H. In another example, each p is 0; X and X³ are each 0; X¹ and X² are each, independently (—CH₂—)ₘ, with m being 1; and R⁴ and R^{4′} are each H, such that the compound of formula (I) is a compound of formula:

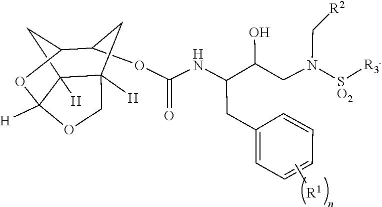

In still another example, G¹ and G² are each (—CH₂—)ₚ, with p being 1; X is (—CH₂—)ₘO—, wherein m is 1; X³ is O; X¹ and X² are each a bond; and R⁴ and R^{4′} are each H, such that the compound of formula (I) is a compound of formula:

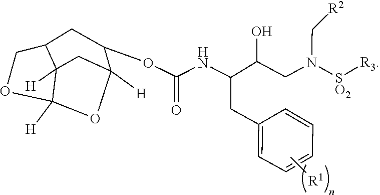

All diastereomers of the compounds of the formula (I) are contemplated herein, including the following:

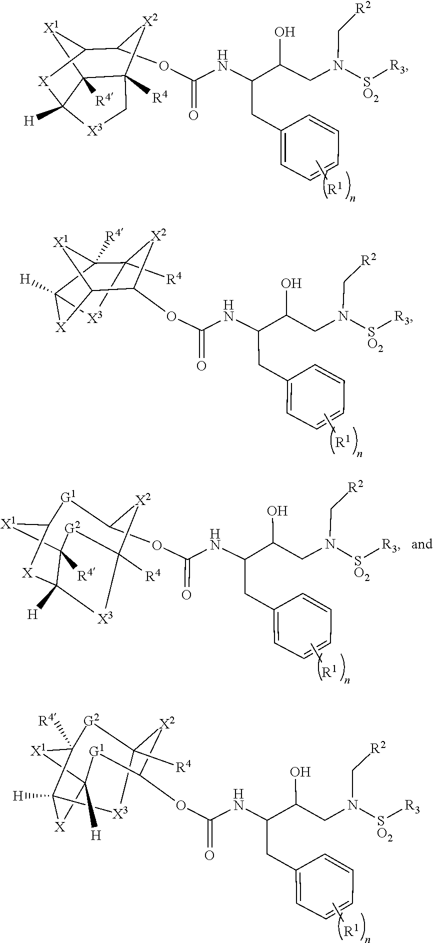

or a pharmaceutically acceptable salt, polymorph, prodrug, solvate or clathrate thereof, wherein: n is an integer from 0 to 3; G¹ and G² are each independently (—CHR⁵—)ₚ, wherein p is 0 or 1 and each R⁵ is independently H or alkyl; X is (—CHR⁵—)ₘO—, wherein m is 0, 1 or 2 and each R⁵ is independently H or alkyl; X³ is (—CHR⁵—)_{d}O—, wherein d is 1 or 2 and each R⁵ is independently H or alkyl; X¹ and X² are each, independently, (—CHR⁵—)ₘ, wherein m is 0, 1 or 2 and each R⁵ is independently H or alkyl; each R¹ is independently alkyl, alkoxy, aryl, heterocyclyl, halo, hydroxy or amino; R² is alkyl; R³ is aryl, benzthiazole, benzoxazole, benzofuranyl or indolyl; and R⁴ is H, alkyl or alkoxy. In some examples, n is 0. In addition, (i) X¹ can be (—CHR⁵—)ₘ, with m being 2, and X² being (—CHR⁵—)ₘ, with m being 1, and each R⁵ being the same or different and as defined herein; (ii) X¹ and X² can be (—CHR⁵—)ₘ, with each m being 1, and each R⁵ being the same or different and as defined herein; (iii) X¹ can be (—CHR⁵—)ₘ, with m being 0, X² can be (—CHR⁵—)ₘ, with m being 1, and each R⁵ being the same or different and as defined herein; or (iv) X¹ and X² can be (—CHR⁵—)ₘ, with m being 0, provided that at least one of G¹ and G² is (—CHR⁵—)ₚ, wherein at least one p is 1 and each R⁵ is independently H or alkyl. In any instance of (i), (ii), (iii) or (iv) X can be O, X³ can be O or X and X³ can be O. In addition, in any instance of (i), (ii), (iii) or (iv) at least one p is 0, such that at least one of G¹ and G² is a bond. In any of the foregoing examples, R⁴ and R^{4′} can each, independently, be H or alkyl. For example, R⁴ can be H. In another example, R^{4′} can be H. In yet another example, R⁴ and R^{4′} are each H. In another example, each p is 0; X and X³ are each 0; X¹ and X² are each independently (—CHR⁵—)ₘ; and R⁴ and R^{4′} are each H. In still another example, each p is 1; X and X³ are each 0; X¹ and X² are each independently (—CHR⁵—)ₘ; and R⁴ and R^{4′} are each H. In another example, each p is 0; X and X³ are each 0; X¹ and X² are each, independently, (—CH₂—)ₘ, with m being 1; and R⁴ and R^{4′} are each H, such that the compound of formula (I) is a compound of formula:

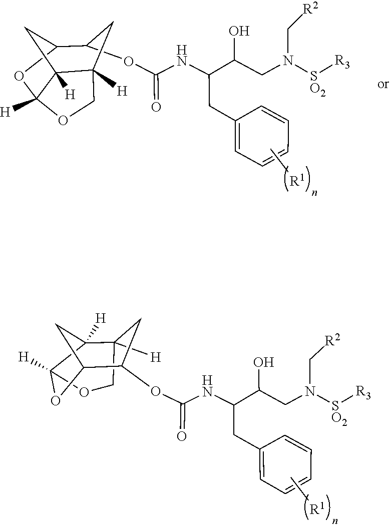

In still another example, G¹ and G² are each (—CH₂—)ₚ, with p being 1; X is (—CH₂—)ₘO—, wherein m is 1; X³ is O; X¹ and X² are each a bond; and R⁴ and R^{4′} are each H, such that the compound of formula (I) is a compound of formula:

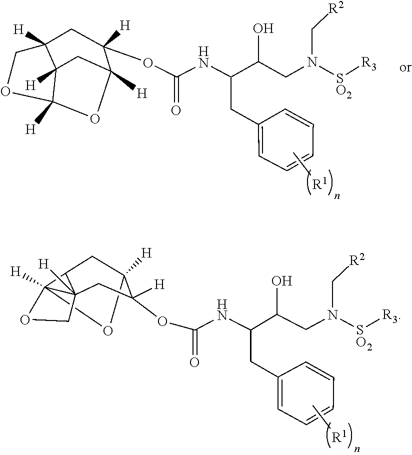

In any of the examples disclosed herein, R³ can be unsubstituted or substituted aryl. R³ can be, for example, phenyl. But R³ can be substituted aryl. The substituted aryl groups represented by R³ herein can be, for example, selected from the group consisting of:

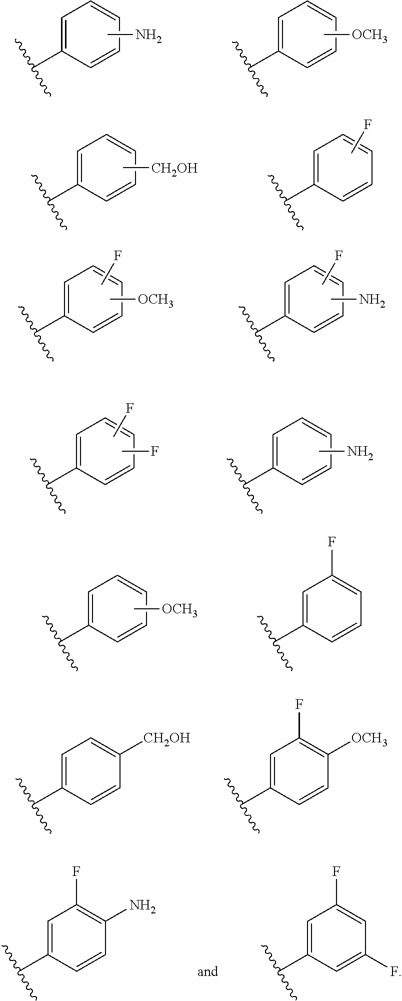

In any of the examples disclosed herein, R³ can be a benzthiazole or a benzoxazole:

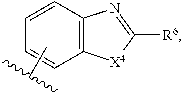

wherein R⁶ is alkyl (e.g., C₁-C₆ alkyl), alkylamino (e.g., C₁-C₆ alkylamino), cycloalkylamino (e.g., C₃-C₆ cycloalkylamino), cycloalkyl heterocycloamino (e.g., C₃-C₆ cycloalkyl-C₃-C₆ heterocycloamino), heterocyclo cycloalkylamino (e.g., C₃-C₆ heterocyclo-C₃-C₆ cycloalkylamino) or heterocycloamino (e.g., C₃-C₆ heterocycloamino); and X⁴ is S, O or NR⁷, wherein R⁷ is H, alklyl, cycloalkyl or alkylaryl. X⁴ can be S or O.

Examples of the compounds of the formula (I) include, but are not limited to, the compounds of formulae:

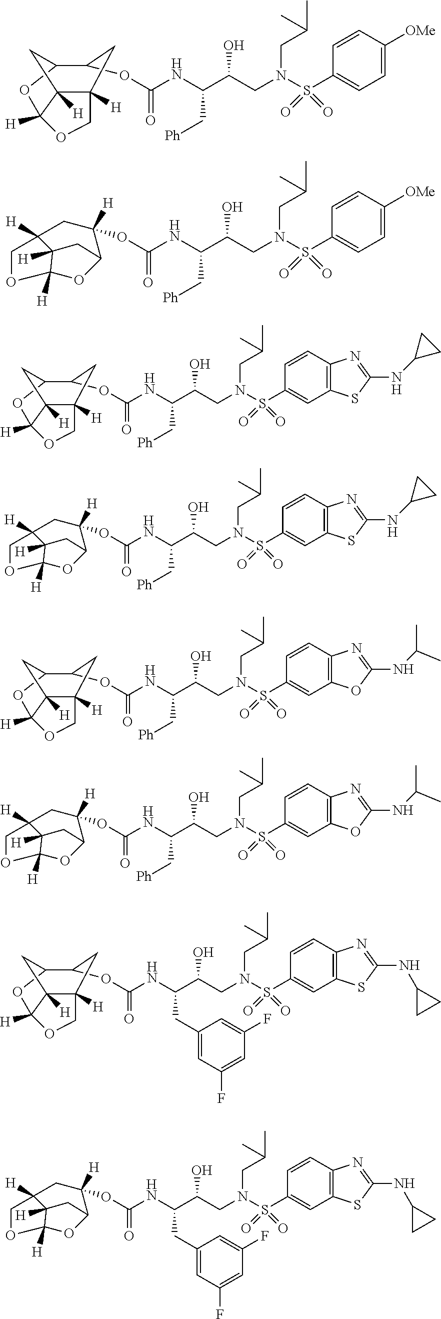

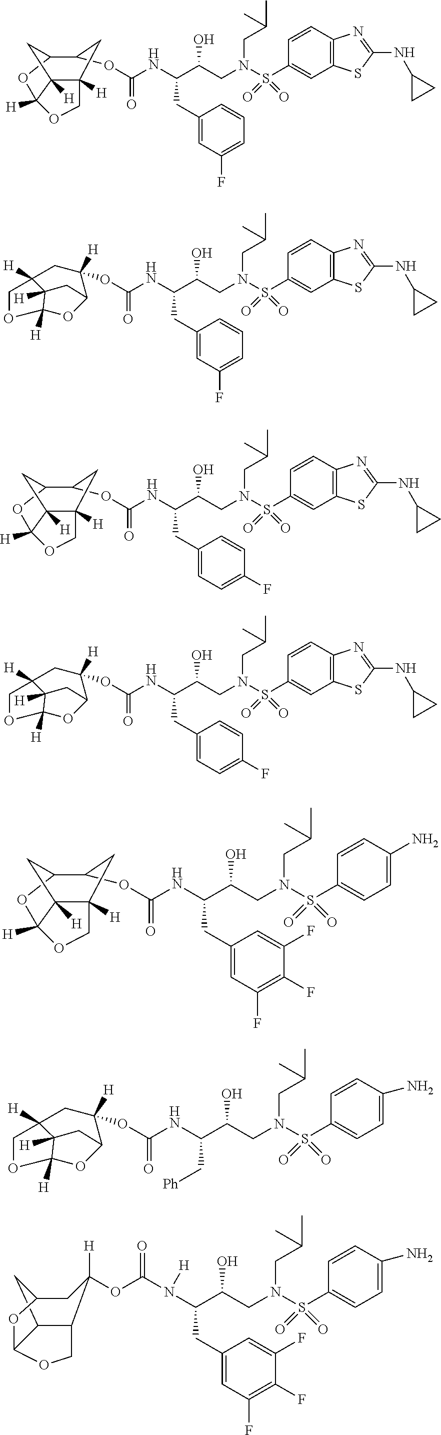

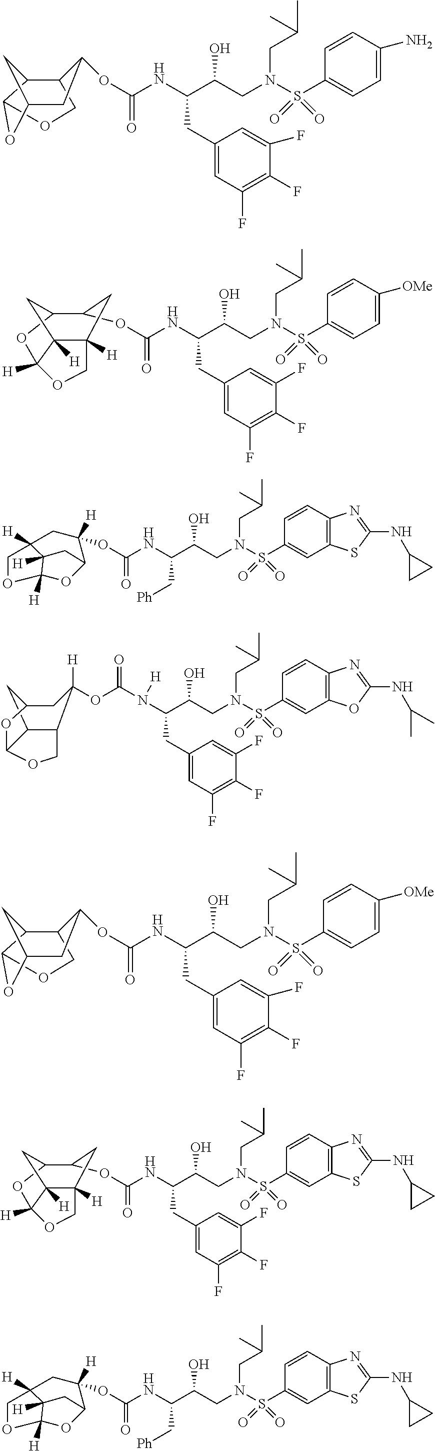

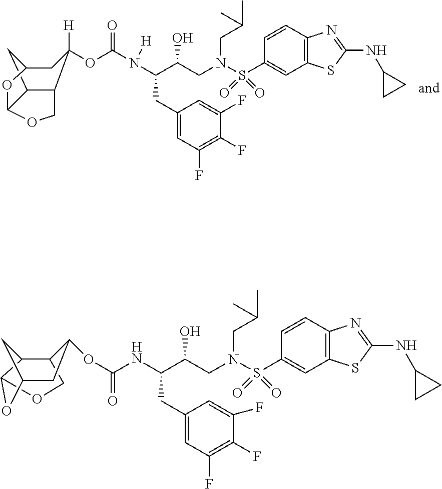 [SMILES: CC(C)CN(C[C@@H](O)[C@H](Cc1cc(F)c(F)c(F)c1)NC(=O)OC1CC2CC3C(OCC13)O2)S(=O)(=O)c1ccc2nc(NC3CC3)sc2c1 ; CC(C)CN(C[C@@H](O)[C@H](Cc1cc(F)c(F)c(F)c1)NC(=O)OC1CC2CC3C(OCC13)O2)S(=O)(=O)c1ccc2nc(NC3CC3)sc2c1]

or a pharmaceutically acceptable salt, polymorph, prodrug, solvate or clathrate thereof.

The disclosure also relates to compounds of the formula (II), which can serve as, among other things, building blocks for the various compounds described herein:

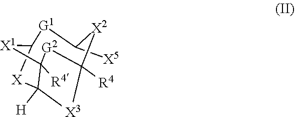

or a pharmaceutically acceptable salt, polymorph, prodrug, solvate or clathrate thereof, wherein the groups X, X¹, X², X³, G¹, G², and R⁴ are as defined herein; and wherein X⁵ is selected from the group consisting of hydroxy, alkoxy, amino, C(O)R, C(O)OR, OC(O)OR, C(O)N(R)₂, OC(O)N(R)₂, C(S)N(R)₂, (CH₂)₀₋₂O(R)C(O)R, (CH₂)₀₋₂N(R)C(O)R, (CH₂)₀₋₂O(R)C(O)OR, (CH₂)₀₋₂O(R)C(O)OR or (CH₂)₀₋₂N(R)N(R)₂, wherein each R can be, independently, hydrogen, alkyl, acyl, cycloalkyl, aryl, aralkyl, heterocyclyl, heteroaryl, or heteroarylalkyl, wherein any alkyl, acyl, cycloalkyl, aryl, aralkyl, heterocyclyl, heteroaryl, or heteroarylalkyl or two R groups bonded to a nitrogen atom or to adjacent nitrogen atoms can together with the nitrogen atom or atoms form a heterocyclyl.

The compound of formula (II) can be a compound of formula (II), wherein:

X¹ is (—CHR⁵—)ₘ, with m being 2, and X² being (—CHR⁵—)ₘ, with m being 1;

X¹ and X² are each (—CHR⁵—)ₘ, with each m being 1;

X¹ is (—CHR⁵—)ₘ, with m being 0, and X² is (—CHR⁵—)ₘ, with m being 1; or

X¹ and X² are each (—CHR⁵—)ₘ, with m being 0, provided that at least one of

G¹ and G² is (—CHR⁵—)ₚ, wherein at least one p is 1.

The compound of formula (II) can be a compound of formula (II), wherein:

X is O;

X³ is O; or

X and X³ are O.

The compound of formula (II) can be a compound of formula (II), wherein at least one p is 0, such that at least one of G¹ and G² is a bond.

The compound of formula (II) can be a compound of formula (II), wherein R⁴ and R^{4′} are each, independently, H or alkyl.

The compound of formula (II) can be a compound of formula (II), wherein:

each p is 0;

X and X³ are each O,

X¹ and X² are each, independently, (—CHR⁵—)ₘ; and

R⁴ and R^{4′} are each H.

The compound of formula (II) can be a compound of formula:

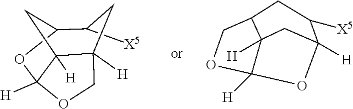

or a pharmaceutically acceptable salt, polymorph, prodrug, solvate or clathrate thereof.

All diastereomers of the compounds of the formula (II) are contemplated herein, including the following:

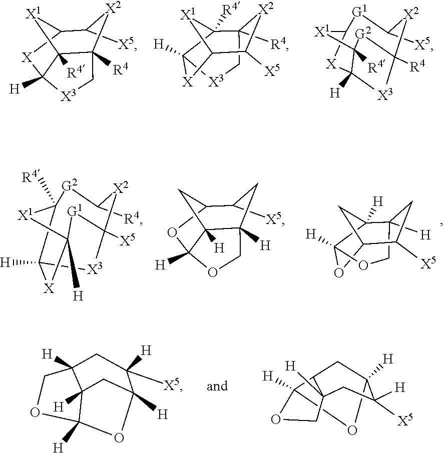

or a pharmaceutically acceptable salt, polymorph, prodrug, solvate or clathrate thereof, wherein X, X¹—X³, X⁵, R⁴, and R^{4′} are defined herein.

### Pharmaceutical Compositions

Pharmaceutical compositions comprising one or more compounds as described herein (e.g., a compound of the formula (I)) and one or more pharmaceutically acceptable carriers, diluents, excipients or combinations thereof are also provided. A “pharmaceutical composition” refers to a chemical or biological composition suitable for administration to a subject (e.g., a mammal, such as a human). Such compositions may be specifically formulated for administration via one or more of a number of routes including, but not limited to, buccal, cutaneous, epicutaneous, epidural, infusion, inhalation, intraarterial, intracardial, intracerebroventricular, intradermal, intramuscular, intranasal, intraocular, intraperitoneal, intraspinal, intrathecal, intravenous, oral, parenteral, pulmonary, rectally via an enema or suppository, subcutaneous, subdermal, sublingual, transdermal, and transmucosal. In addition, administration can be by means of a capsule, drops, foam, gel, gum, injection, liquid, patch, pill, porous pouch, powder, tablet, or other suitable means of administration.

A “pharmaceutical excipient” or a “pharmaceutically acceptable excipient” comprises a carrier, sometimes a liquid, in which an active therapeutic agent is formulated. The excipient generally does not provide any pharmacological activity to the formulation, though it may provide chemical and/or biological stability, and release characteristics. Examples of suitable formulations can be found, for example, in Remington, The Science And Practice of Pharmacy, 20th Edition, (Gennaro, A. R., Chief Editor), Philadelphia College of Pharmacy and Science, 2000, which is incorporated by reference in its entirety.

As used herein “pharmaceutically acceptable carrier” or “excipient” includes any and all solvents, dispersion media, coatings, antibacterial and antifungal agents, and isotonic and absorption delaying agents that are physiologically compatible. In one embodiment, the carrier is suitable for parenteral administration. Alternatively, the carrier can be suitable for intravenous, intraperitoneal, intramuscular, sublingual, or oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active compound, use thereof in the pharmaceutical compositions of the invention is contemplated. Supplementary active compounds can also be incorporated into the compositions.

Pharmaceutical compositions may be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (e.g., glycerol, propylene glycol, and liquid polyethylene glycol), and suitable mixtures thereof. The proper fluidity can be maintained, for example, by the use of a coating such as lecithin, by the maintenance of the required particle size in the case of dispersion and by the use of surfactants.

In some cases isotonic agents can be included in the pharmaceutical compositions. For example, sugars, polyalcohols such as mannitol, sorbitol, or sodium chloride can be included in the composition. Prolonged absorption of the injectable compositions can be brought about by including in the composition an agent which delays absorption, such as monostearate salts and gelatin. Moreover, the compounds described herein can be formulated in a time-release formulation, for example in a composition that includes a slow-release polymer. The active compounds can be prepared with carriers that will protect the compound against rapid release, such as a controlled release formulation, including implants and microencapsulated delivery systems. Biodegradable, biocompatible polymers may be used, such as ethylene vinyl acetate, polyanhydrides, polyglycolic acid, collagen, polyorthoesters, polylactic acid and polylactic, polyglycolic copolymers (PLG). Many methods for the preparation of such formulations are known to those skilled in the art.

Oral forms of administration are also contemplated herein. The pharmaceutical compositions may be orally administered as a capsule (hard or soft), tablet (film-coated, enteric-coated or uncoated), powder or granules (coated or uncoated) or liquid (solution or suspension). The formulations can be conveniently prepared by any of the methods well-known in the art. The pharmaceutical compositions can include one or more suitable production aids or excipients, including fillers, binders, disintegrants, lubricants, diluents, flow agents, buffering agents, moistening agents, preservatives, colorants, sweeteners, flavors, and pharmaceutically compatible carriers.

For each of the recited embodiments, the compounds can be administered by a variety of dosage forms as known in the art. Any biologically-acceptable dosage form known to persons of ordinary skill in the art, and combinations thereof, are contemplated. Examples of such dosage forms include, without limitation, chewable tablets, quick-dissolve tablets, effervescent tablets, reconstitutable powders, elixirs, liquids, solutions, suspensions, emulsions, tablets, multi-layer tablets, bi-layer tablets, capsules, soft gelatin capsules, hard gelatin capsules, caplets, lozenges, chewable lozenges, beads, powders, gum, granules, particles, microparticles, dispersible granules, cachets, douches, suppositories, creams, topicals, inhalants, aerosol inhalants, patches, particle inhalants, implants, depot implants, ingestibles, injectables (including subcutaneous, intramuscular, intravenous, and intradermal), infusions, and combinations thereof.

Other compounds, which can be included by admixture, are, for example, medically inert ingredients (e.g., solid and liquid diluent), such as lactose, dextrosesaccharose, cellulose, starch or calcium phosphate for tablets or capsules, olive oil or ethyl oleate for soft capsules, and water or vegetable oil for suspensions or emulsions; lubricating agents, such as silica, talc, stearic acid, magnesium or calcium stearate and/or polyethylene glycols; gelling agents, such as colloidal clays; thickening agents, such as gum tragacanth or sodium alginate, binding agents, such as starches, arabic gums, gelatin, methylcellulose, carboxymethylcellulose or polyvinylpyrrolidone; disintegrating agents, such as starch, alginic acid, alginates or sodium starch glycolate; effervescing mixtures; dyestuff; sweeteners; wetting agents, such as lecithin, polysorbates or laurylsulphates; and other therapeutically acceptable accessory ingredients, such as humectants, preservatives, buffers and antioxidants, which are known additives for such formulations.

Liquid dispersions for oral administration can be syrups, emulsions, solutions, or suspensions. The syrups can contain as a carrier, for example, saccharose or saccharose with glycerol and/or mannitol and/or sorbitol. The suspensions and the emulsions can contain a carrier, for example a natural gum, agar, sodium alginate, pectin, methylcellulose, carboxymethylcellulose, or polyvinyl alcohol.

The amount of active compound in a therapeutic composition according to various embodiments may vary according to factors such as the disease state, age, gender, weight, patient history, risk factors, predisposition to disease, administration route, pre-existing treatment regime (e.g., possible interactions with other medications), and weight of the individual. Dosage regimens can be adjusted to provide the optimum therapeutic response. For example, a single bolus can be administered, several divided doses can be administered over time, or the dose can be proportionally reduced or increased as indicated by the exigencies of therapeutic situation.

“Dosage unit form,” as used herein, refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of sensitivity in individuals. In therapeutic use for treatment of conditions in mammals (e.g., humans) for which the compounds of the various embodiments described herein or an appropriate pharmaceutical composition thereof are effective, the compounds of the various embodiments described herein can be administered in an effective amount. The dosages as suitable for this invention can be a composition, a pharmaceutical composition or any other compositions described herein.

The dosage can be administered once, twice, or thrice a day, although more frequent dosing intervals are possible. The dosage can be administered every day, every 2 days, every 3 days, every 4 days, every 5 days, every 6 days, and/or every 7 days (once a week). In one embodiment, the dosage can be administered daily for up to and including 30 days, preferably between 7-10 days. In another embodiment, the dosage can be administered twice a day for 10 days. If the patient requires treatment for a chronic disease or condition, the dosage can be administered for as long as signs and/or symptoms persist. The patient may require “maintenance treatment” where the patient is receiving dosages every day for months, years, or the remainder of their lives. In addition, the composition can be administered to effect prophylaxis of recurring symptoms. For example, the dosage can be administered once or twice a day to prevent the onset of symptoms in patients at risk, especially for asymptomatic patients.

The compositions described herein may be administered in any of the following routes: buccal, epicutaneous, epidural, infusion, inhalation, intraarterial, intracardial, intracerebroventricular, intradermal, intramuscular, intranasal, intraocular, intraperitoneal, intraspinal, intrathecal, intravenous, oral, parenteral, pulmonary, rectally via an enema or suppository, subcutaneous, subdermal, sublingual, transdermal, and transmucosal. The preferred routes of administration are buccal and oral. The administration can be local, where the composition is administered directly, close to, in the locality, near, at, about, or in the vicinity of, the site(s) of disease, e.g., inflammation, or systemic, wherein the composition is given to the patient and passes through the body widely, thereby reaching the site(s) of disease. Local administration can be administration to the cell, tissue, organ, and/or organ system, which encompasses and/or is affected by the disease, and/or where the disease signs and/or symptoms are active or are likely to occur. Administration can be topical with a local effect, i.e., the composition can be applied directly where its action is desired. Administration can be enteral wherein the desired effect is systemic (non-local), e.g., the composition can be delivered via the digestive tract. Administration can be parenteral, where the desired effect is systemic, e.g., the composition is delivered by routes other than the digestive tract.

### Methods of Treatment

Compositions comprising a therapeutically effective amount of one or more compounds of the various embodiments described herein (e.g., a compound of the formula (I)) are also contemplated. The compositions are useful in a method for treating an HIV (e.g., HIV-1) infection or AIDS, the method comprising administering a therapeutically effective amount of one or more compounds described herein to a patient in need thereof. Also contemplated herein is one or more compounds described herein for use as a medicament for treating a patient in need of relief from an HIV infection or AIDS.

The term “effective amount” or “therapeutically effective amount” as used herein, refers to that amount of one or more compounds of the various embodiments described herein (e.g. a compound of the formula (I)) that elicits a biological or medicinal response in a tissue system, animal or human, that is being sought by a researcher, veterinarian, medical doctor or other clinician, which includes alleviation of the symptoms of the disease or disorder being treated. In some embodiments, the therapeutically effective amount is that which may treat or alleviate the disease or symptoms of the disease at a reasonable benefit/risk ratio applicable to any medical treatment. However, it is to be understood that the total daily usage of the compounds and compositions described herein may be decided by the attending physician within the scope of sound medical judgment. The specific therapeutically-effective dose level for any particular patient will depend upon a variety of factors, including the condition being treated and the severity of the condition; activity of the specific compound employed; the specific composition employed; the age, body weight, general health, gender and diet of the patient; the time of administration, route of administration, and rate of excretion of the specific compound employed; the duration of the treatment; drugs used in combination or coincidentally with the specific compound employed; and like factors well-known to the researcher, veterinarian, medical doctor or other clinician. It is also appreciated that the therapeutically effective amount can be selected with reference to any toxicity, or other undesirable side effect, that might occur during administration of one or more of the compounds described herein.

The compounds described herein can have an HIV-1 protease inhibition constant (Kᵢ) of from about 1 fM to about 200 nM (e.g., about 100 fM to about 200 nM, about 100 fM to about 100 pM, about 250 fM to about 100 pM, about 500 fM to about 5 pM, about 5 pM to about 100 pM, about 50 pM to about 250 pM, about 500 pM to about 100 nM or about 300 pM to about 75 nM).

Alternatively, or in addition, compounds described herein have an antiviral activity in vitro against a wild-type laboratory strain, HIV-1_{LAI}, with half-maximal inhibitory concentration (IC₅₀) of from about 1 fM to about 200 nM (e.g., about 100 fM to about 200 nM, about 100 fM to about 100 pM, about 250 fM to about 100 pM, about 500 fM to about 5 pM, from about 10 pM to about 50 nM, about 10 pM to about 500 pM, about 100 pM to about 750 pM, about 500 pM to about 1 nM or about 500 pM to about 50 nM).

Alternatively, or in addition, compounds described herein have a darunavir-resistant HIV-1 variant (e.g., NL4-3R, DRV_{R}P20, DRV_{R}P30, and DRV_{R}P51) antiviral IC₅₀ of from about 200 fM to about 100 nM (e.g., from about 200 fM to about 600 fM, about 200 fM to about 50 pM, about 500 fM to about 500 pM, about 300 fM to about 1 pM).

Alternatively, or in addition, compounds described herein have a darunavir-resistant HIV-1 variants (e.g., NL4-3R, DRV_{R}P20, DRV_{R}P30, and DRV_{R}P51) IC₅₀ of from about 50 pM to about 50 nM (e.g., from about 100 pM to about 50 nM or about 500 pM to about 10 nM). In still other embodiments, the compounds of the various embodiments described herein have a darunavir-resistant HIV-1 protease (e.g., NL4-3R, DRV_{R}P20, DRV_{R}P30, and DRV_{R}P51) antiviral IC₅₀ of from about 1 nM to about 100 nM (e.g., from about 10 nM to about 75 nM or about 10 nM to about 75 nM).

Values expressed in a range format should be interpreted in a flexible manner to include not only the numerical values explicitly recited as the limits of the range, but also to include all the individual numerical values or sub-ranges encompassed within that range as if each numerical value and sub-range were explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

### Additional Definitions

In this document, the terms “a,” “an,” or “the” are used to include one or more than one unless the context clearly dictates otherwise. The term “or” is used to refer to a nonexclusive “or” unless otherwise indicated. The invention illustratively described herein may be suitably practiced in the absence of any element(s) or limitation(s), which is/are not specifically disclosed herein. Thus, for example, each instance herein of any of the terms “comprising,” “consisting essentially of,” and “consisting of” may be replaced with either of the other two terms. In addition, it is to be understood that the phraseology or terminology employed herein, and not otherwise defined, is for the purpose of description only and not of limitation. Any use of section headings is intended to aid reading of the document and is not to be interpreted as limiting. Further, information that is relevant to a section heading may occur within or outside of that particular section. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

In the methods described herein, the steps can be carried out in any order without departing from the principles of the invention, except when a temporal or operational sequence is explicitly recited. Furthermore, specified steps can be carried out concurrently unless explicit claim language recites that they be carried out separately. For example, a claimed step of doing X and a claimed step of doing Y can be conducted simultaneously within a single operation, and the resulting process will fall within the literal scope of the claimed process.

The term “about” can allow for a degree of variability in a value or range, for example, within 10%, within 5%, or within 1% of a stated value or of a stated limit of a range.

The term “substantially” refers to a majority of, or mostly, as in at least about 50%, 60%, 70%, 80%, 90%, 95%, 96%, 97%, 98%, 99%, 99.5%, 99.9%, 99.99%, or at least about 99.999% or more.

The term “substituted” or “substituent” refers to a group that can be or is substituted onto a molecule or onto another group (e.g., on an aryl or an alkyl group). Examples of substituents include, but are not limited to, a halogen (e.g., F, Cl, Br, and I), OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, —(CH₂)₀₋₂P(O)(OR)₂, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)C(O)OR, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, or C(═NOR)R wherein each R can be, independently, hydrogen, alkyl, acyl, cycloalkyl, aryl, aralkyl, heterocyclyl, heteroaryl, or heteroarylalkyl, wherein any alkyl, acyl, cycloalkyl, aryl, aralkyl, heterocyclyl, heteroaryl, or heteroarylalkyl or two R groups bonded to a nitrogen atom or to adjacent nitrogen atoms can together with the nitrogen atom or atoms form a heterocyclyl, which can be mono- or independently multi-substituted.

The term “alkyl” refers to substituted or unsubstituted straight chain and branched alkyl groups and cycloalkyl groups having from 1 to 40 carbon atoms (C₁-C₄₀), 1 to about 20 carbon atoms (C₁-C₂₀), 1 to 12 carbons (C₁-C₁₂), 1 to 8 carbon atoms (C₁—C), or, in some embodiments, from 1 to 6 carbon atoms (C₁-C₆). Examples of straight-chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. The term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “cycloalkyl” refers to substituted or unsubstituted cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. In some embodiments, cycloalkyl groups can have 3 to 6 carbon atoms (C₃-C₆). Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like.

The term “cycloalkylalkyl” refers to substituted or unsubstituted alkyl groups as defined herein in which a hydrogen or carbon bond of an alkyl group as defined herein is replaced with a bond to a cycloalkyl group as defined herein. Representative cycloalkylalkyl groups include, but are not limited to, cyclopentylalkyl.

The term “acyl” refers to a group containing a carbonyl moiety wherein the group is bonded via the carbonyl carbon atom. The carbonyl carbon atom is also bonded to another carbon atom, which can be part of a substituted or unsubstituted alkyl, aryl, aralkyl cycloalkyl, cycloalkylalkyl, heterocyclyl, heterocyclylalkyl, heteroaryl, heteroarylalkyl group or the like. In the special case wherein the carbonyl carbon atom is bonded to a hydrogen, the group is a “formyl” group, an acyl group as the term is defined herein. An acyl group can include 0 to about 12-40, 6-10, 1-5 or 2-5 additional carbon atoms bonded to the carbonyl group. An acryloyl group is an example of an acyl group. An acyl group can also include heteroatoms within the meaning here. A nicotinoyl group (pyridyl-3-carbonyl) is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “aryl” refers to substituted or unsubstituted cyclic aromatic hydrocarbons that do not contain heteroatoms in the ring. Thus, aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons (C₆-C₁₄) or from 6 to 10 carbon atoms (C₆-C₁₀) in the ring portions of the groups. Aryl groups can be unsubstituted or substituted, as defined herein. Representative substituted aryl groups can be mono-substituted or substituted more than once, such as, but not limited to, 2-, 3-, 4-, 5-, or 6-substituted phenyl or 2-8 substituted naphthyl groups, which can be substituted with carbon or non-carbon groups such as those listed herein.

The term “aralkyl” and “arylalkyl” refers to alkyl groups as defined herein in which a hydrogen or carbon bond of an alkyl group is replaced with a bond to an aryl group as defined herein. Representative aralkyl groups include benzyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

The term “heterocyclyl” or “heterocyclo” refers to substituted or unsubstituted aromatic and non-aromatic ring compounds containing 3 or more ring members, of which, one or more (e.g., 1, 2 or 3) is a heteroatom such as, but not limited to, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. In some embodiments, heterocyclyl groups include heterocyclyl groups that include 3 to 8 carbon atoms (C₃-C₈), 3 to 6 carbon atoms (C₃-C₆), 3 to 5 carbon atoms (C₃-C₅) or 6 to 8 carbon atoms (C₆-C₈). A heterocyclyl group designated as a C₂-heterocyclyl can be a 5-ring with two carbon atoms and three heteroatoms, a 6-ring with two carbon atoms and four heteroatoms and so forth. Likewise a C₄-heterocyclyl can be a 5-ring with one heteroatom, a 6-ring with two heteroatoms, and so forth. The number of carbon atoms plus the number of heteroatoms equals the total number of ring atoms. A heterocyclyl ring can also include one or more double bonds. A heteroaryl ring is an embodiment of a heterocyclyl group. The phrase “heterocyclyl group” includes fused ring species including those that include fused aromatic and non-aromatic groups. Representative heterocyclyl groups include, but are not limited to, pyrrolidinyl, azetidinyl, piperidynyl, piperazinyl, morpholinyl, chromanyl, indolinonyl, isoindolinonyl, furanyl, pyrrolidinyl, pyridinyl, pyrazinyl, pyrimidinyl, triazinyl, thiophenyl, tetrahydrofuranyl, pyrrolyl, oxazolyl, oxadiazolyl, imidazolyl, triazyolyl, tetrazolyl, benzoxazolinyl, benzthiazolinyl, and benzimidazolinyl groups. Examples of indolinonyl groups include groups having the general formula:

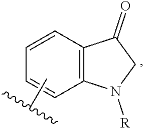

wherein R is as defined herein.
Examples of isoindolinonyl groups include groups having the general formula:

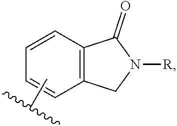

wherein R is as defined herein.
Examples of benzoxazolinyl groups include groups having the general formula:

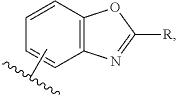

wherein R is as defined herein.
Examples of benzthiazolinyl groups include groups having the general formula:

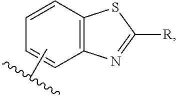

wherein R is as defined herein.
In some embodiments, the group R in benzoxazolinyl and benzthiazolinyl groups is an N(R)₂ group. In some embodiments, each R is hydrogen or alkyl, wherein the alkyl group is substituted or unsubstituted. In some embodiments, the alkyl group is substituted with a heterocyclyl group (e.g., with a pyrrolidinyl group).

The term “heterocyclylalkyl” refers to alkyl groups as defined herein in which a hydrogen or carbon bond of an alkyl group as defined herein is replaced with a bond to a heterocyclyl group as defined herein. Representative heterocyclylalkyl groups include, but are not limited to, furan-2-yl methyl, furan-3-yl methyl, pyridine-3-yl methyl, tetrahydrofuran-2-yl methyl, and indol-2-yl propyl.

The term “heterocyclylalkoxy” refers to alkyl groups in which a hydrogen or carbon bond of an alkyl group is replaced with a bond to a heterocyclyl group and the alkyl group is attached to an oxygen. Representative heterocyclylalkoxy groups include, but are not limited to, —O—(CH₂)_{q}heterocyclyl, wherein q is an integer from 1 to 5. In some embodiments, heterocyclylalkoxy groups include —O—(CH₂)_{q}morpholinyl such as —O—CH₂CH₂-morpholine.

The term “heteroarylalkyl” refers to alkyl groups in which a hydrogen or carbon bond of an alkyl group is replaced with a bond to a heteroaryl group as defined herein.

The term “alkoxy” refers to an oxygen atom connected to an alkyl group, including a cycloalkyl group, as are defined herein. Examples of linear alkoxy groups include, but are not limited to, methoxy, ethoxy, propoxy, butoxy, pentyloxy, hexyloxy, and the like. Examples of branched alkoxy include, but are not limited to, isopropoxy, sec-butoxy, tert-butoxy, isopentyloxy, isohexyloxy, and the like. Examples of cyclic alkoxy include, but are not limited to, cyclopropyloxy, cyclobutyloxy, cyclopentyloxy, cyclohexyloxy, and the like. An alkoxy group can include one to about 12-20 or about 12-40 carbon atoms bonded to the oxygen atom, and can further include double or triple bonds, and can also include heteroatoms. For example, an allyloxy group is an alkoxy group. A methoxyethoxy group is also an alkoxy group, as is a methylenedioxy group in a context where two adjacent atoms of a structure are substituted therewith.

The term “amine” refers to primary, secondary, and tertiary amines having, e.g., the formula N(group)₃ wherein each group can independently be H or non-H, such as alkyl, aryl, and the like. Amines include, but are not limited to R—NH₂, such as, for example, alkylamines; arylamines; alkylarylamines; R₂NH wherein R is defined herein, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

The term “amino group” refers to a substituent of the form —NH₂, —NHR, —NR₂, —NR₃+, wherein each R is defined herein, and protonated forms of each, except for —NR₃+, which cannot be protonated. Accordingly, any compound substituted with an amino group can be viewed as an amine. An “amino group” can be a primary, secondary, tertiary, or quaternary amino group. An “alkylamino” group includes a monoalkylamino, a dialkylamino, and a trialkylamino group.

An example of a “alkylamino” is —NH-alkyl and —N(alkyl)₂.

Examples of a “cycloalkylamino” group are —NH-cycloalkyl and —N(cycloalkyl)₂.

An example of a “cycloalkyl heterocycloamino” group is —NH-(heterocyclo cycloalkyl), wherein the heterocyclo group is attached to the nitrogen and the cycloalkyl group is attached to the heterocyclo group.

An example of a “heterocyclo cycloamino” group is —NH-(cycloalkyl heterocycle), wherein the cycloalkyl group is attached to the nitrogen and the heterocyclo group is attached to the cycloalkyl group.

The terms “halo,” “halogen,” and “halide” group, by themselves or as part of another substituent, mean, unless otherwise stated, a fluorine, chlorine, bromine, or iodine atom.

The term “haloalkyl” group includes mono-halo alkyl groups and poly-halo alkyl groups, in which the halo atoms can be the same or different, and per-halo alkyl groups, in which all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, —CF(CH₃)₂ and the like.

The terms “salts” and “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic groups, such as amines; and alkali or organic salts of acidic groups, such as carboxylic acids. Pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids, such as hydrochloric, hydrobromic, sulfuric, sulfamic, phosphoric, and nitric; and the salts prepared from organic acids, such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic, and the like.

Pharmaceutically acceptable salts can be synthesized from the parent compound, which contains a basic or acidic moiety, by conventional chemical methods. In some instances, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, the disclosure of which is hereby incorporated by reference.

The term “solvate” means a compound, or a salt thereof, that further includes a stoichiometric or non-stoichiometric amount of solvent bound by non-covalent intermolecular forces. Where the solvent is water, the solvate is a hydrate.

The term “clathrate” means a chemical substance consisting of a lattice that traps or contains molecules. Clathrate can be polymeric, or can be host-guest complexes and inclusion compounds. Clathrates can be inclusion compounds in which the guest molecule is in a cage formed by the host molecule or by a lattice of host molecules.

The term “polymorph” refers to a specific form of a compound, for example, polymorphs may represent crystalline forms that can vary in pharmaceutically relevant physical properties between one form and another, for example under different crystallization conditions, environmental conditions, hygroscopic activity of the compounds, etc.

The term “prodrug” means a derivative of a compound that can hydrolyze, oxidize, or otherwise react under biological conditions (in vitro or in vivo) to provide an active compound. Examples of prodrugs include, but are not limited to, derivatives and metabolites of a compound that include biohydrolyzable moieties, such as biohydrolyzable amides, biohydrolyzable esters, biohydrolyzable carbamates, biohydrolyzable carbonates, biohydrolyzable ureides, and biohydrolyzable phosphate analogues. Specific prodrugs of compounds with carboxyl functional groups are the lower alkyl esters of the carboxylic acid. The carboxylate esters are conveniently formed by esterifying any of the carboxylic acid moieties present on the molecule. Prodrugs can typically be prepared using well-known methods, such as those described by Burger's Medicinal Chemistry and Drug Discovery 6th ed. (Donald J. Abraham ed., 2001, Wiley) and Design and Application of Prodrugs (H. Bundgaard ed., 1985, Harwood Academic Publishers GmbH).

The term “patient” or “subject” refers to a mammal suffering from a disease, disorder, or condition. A patient or subject can be a primate, canine, feline, or equine. A patient or subject can be a bird. The bird can be a domesticated bird, such as a chicken. The bird can be a fowl. A patient or subject can be a human.

All patents, patent application publications, journal articles, textbooks, and other publications mentioned in the specification are indicative of the level of skill of those in the art to which the disclosure pertains. All such publications are incorporated herein by reference to the same extent as if each individual publication were specifically and individually indicated to be incorporated by reference.

### EXAMPLES

The present invention can be better understood by reference to the following examples, which are offered by way of illustration. The present invention is not limited to the examples given herein.

### INTRODUCTION

The synthetic strategy for optically active synthesis for the various ligands described herein is shown in Scheme 1. Enantiomeric ligand 6 can be obtained from the functionalized cyclohexane-1,2-diol derivative 7. Structure 7 can be obtained from cyclohexene derivative 9 by asymmetric dihydroxylation reaction. Optically active aldehyde derivative can be derived conveniently from meso diol derivative 8 by enzymatic desymmetrization as the key reaction. The meso-diol 9 can be derived from commercially available and inexpensive 1,2,3,6-tetrahydrophthalic anhydride 10.

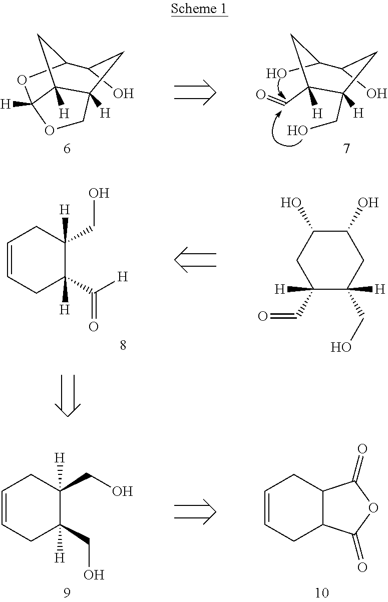

The synthesis of optically active ligand 6 is shown in Scheme 2. Meso-1,2,3,6-tetrahydrophthalic anhydride 10 was reduced by LiAlH₄ in THE at 0° C. for 3 h to provide meso-diol derivative 9 in a multigram scale. Diol 9 was subjected to enzymatic desymmetrization reaction using porcine pancreatic lipase (PPL) in ethyl acetate at 23° C. for 12 h to provide monoacetate derivative 11 in gram scale in 82% yield and 95% ee as determined by HPLC analysis.³⁷ Swern oxidation of alcohol 11 provided aldehyde which was reacted with trimethylorthoformate in the presence of a catalytic amount of tetrabutylammonium bromide (TBAB) at 23° C. for 8 h to provide dimethylacetal derivative 12 in 76% yield over two steps. For diastereoselective dihydroxylation, we carried out Sharpless asymmetric dihydroxylation using AD-mix-β. Thus, reaction of 12 with AD-mix-β in a mixture (1:1) of t-butanol and water at 0° C. to 23° C. for 24 h resulted in a 1:1 mixture of diastereomeric diol. The resulting diol was subjected to saponification using 1 N aqueous NaOH in MeOH at 0° C. to 23° C. for 3 h to provide triol derivatives 13 and 14 in 90% yield over two-steps. These triol derivatives were separated by silica gel chromatography using 5% MeOH in CH₂Cl₂ as the eluent. Triol derivative 13 was reacted with a catalytic amount of camphorsulfonic acid (CSA) in CH₂Cl₂ at 0° C. for 1 h to provide optically active tricyclic ligand alcohol 6 in 82% yield.

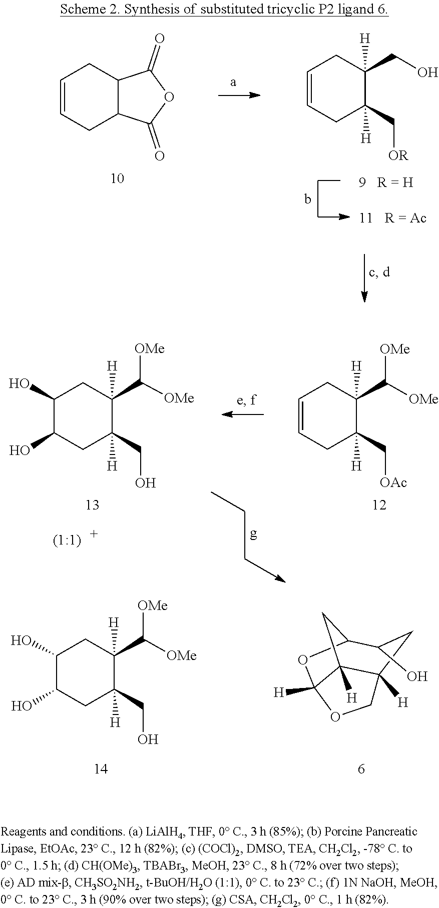

The synthesis of enantiomeric ligand ent-6 from meso-diol 10 is shown in Scheme 3. Diol 10 was converted to diacetate derivative 15 by reaction with acetic anhydride and pyridine in the presence of a catalytic amount of DMAP at 23° C. for 16 h. Exposure of diacetate to PPL in 0.1 M phosphate buffer at pH 7 in the presence of aqueous NaHCO₃ at 23° C. for 16 h provided optically active alcohol 16 in 84% yield. Alcohol 16 was converted to dimethylacetal 17 in 80% yield as described above. Exposure of dimethylacetal 17 to Sharpless asymmetric dihydroxylation reaction with AD-mix-β afforded a 1:1 mixture of diastereomeric diol.³⁴ Deprotection of the acetate derivative furnished triol derivatives 18 and 19 which were separated by silica gel chromatography. Treatment of the major triol derivative 18 with a catalytic amount of CSA in CH₂Cl₂ afforded ligand alcohol ent-6 in 79% yield.

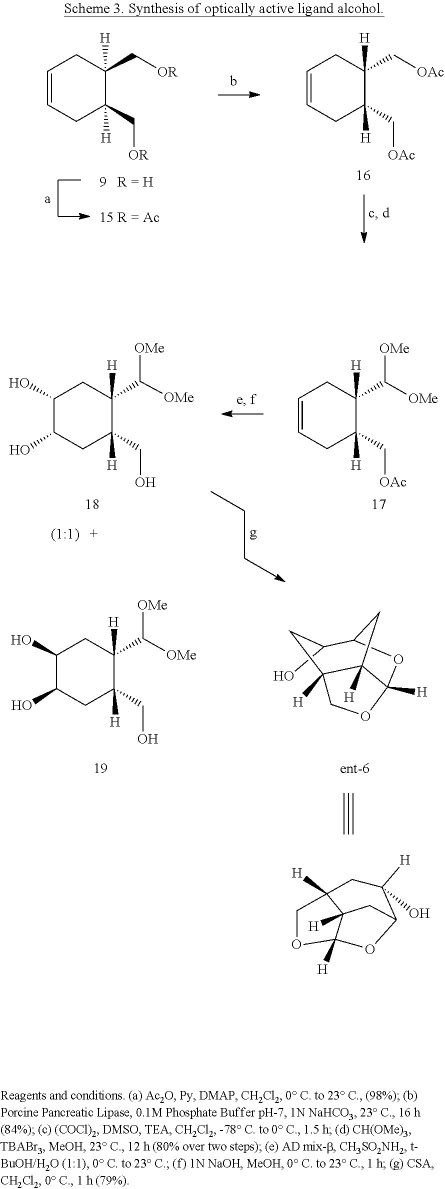

The synthesis of the designed protease inhibitors was carried out in a two-step sequence involving synthesis of activated carbonates followed by reaction of these carbonates with appropriate hydroxyethylaminesulfonamide isosteres. The syntheses of various activated carbonates are shown in Scheme 4. Optically active ligand alcohols 6 and ent-6 synthesized above were converted to their respective activated carbonates 19 and 20. As shown, reaction of ligand alcohols 6 and ent-6 with 4-nitrophenylchloroformate in the presence of pyridine in CH₂Cl₂ at 0° C. to 23° C. for 12 h provided activated carbonates 19 and 20 in 87% and 88% yields, respectively. These carbonates were then converted to urethane derivatives using amines 21-26. The synthesis of various inhibitors containing Chf-THF as the P2 ligands on the hydroxyethylamine sulfonamide isostere is shown in Scheme 5. Reactions of activated carbonates 6 with known amine derivatives 21-26 in the presence of diisopropylethylamine (DIPEA) in CH₃CN at 23° C. for 72 h provided inhibitors 4a-f in good yields (65-86%). Similarly, reactions of carbonate ent-6 with amines 21-26 under similar conditions afforded inhibitors 5a-5f in very good yields (59-87%).

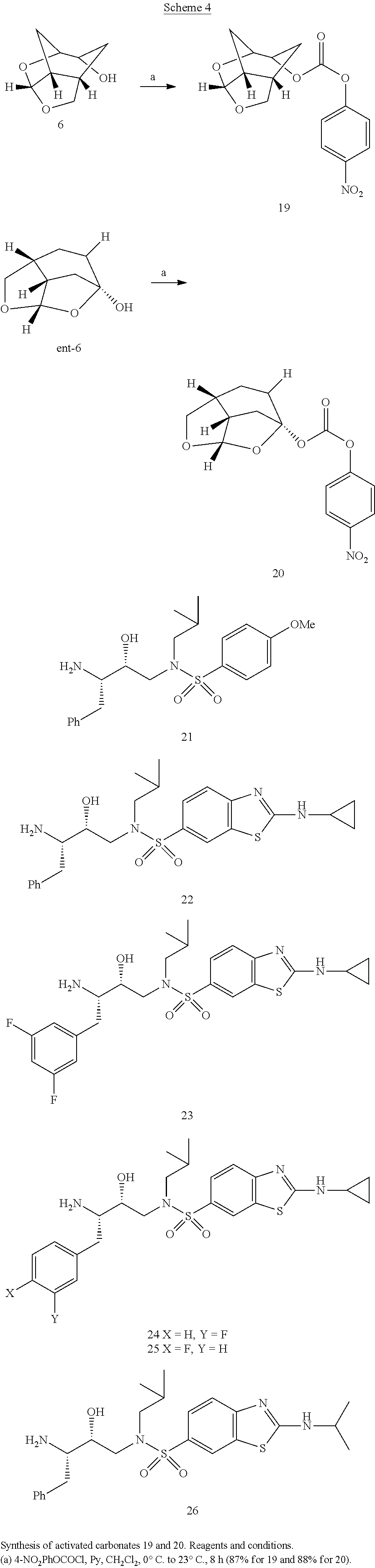

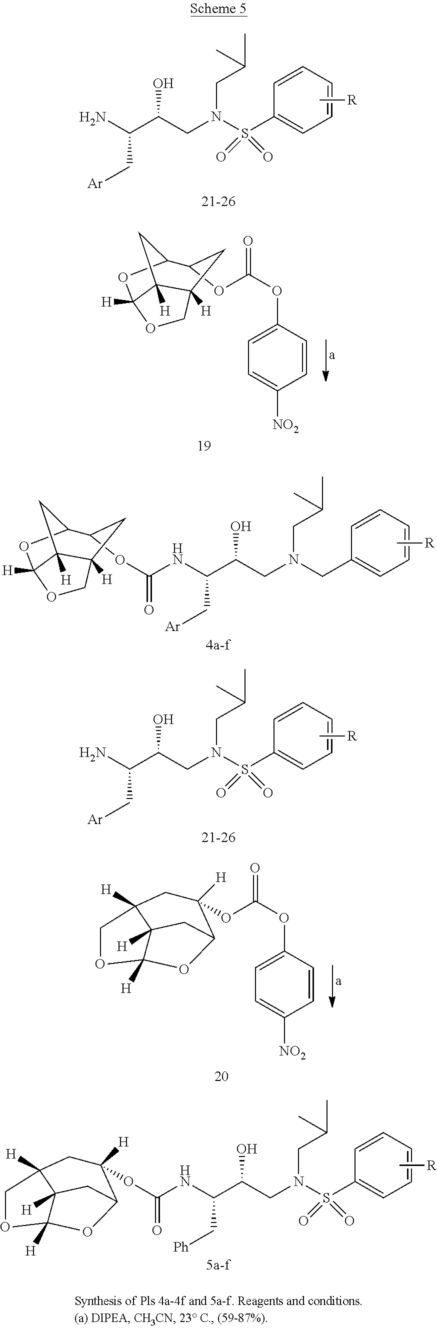

### EXPERIMENTAL

All chemicals and reagents were purchased from commercial suppliers and used without further purification unless otherwise noted. The following reaction solvents were distilled prior to use: dichloromethane from calcium hydride, diethyl ether and tetrahydrofuran from Na/benzophenone, methanol and ethanol from activated magnesium under argon. All reactions were carried out under an argon atmosphere in either flame or oven-dried (120° C.) glassware. TLC analysis was conducted using glass-backed Thin-Layer Silica Gel Chromatography Plates (60 Å, 250 μm thickness, F-254 indicator). Column chromatography was performed using 230-400 mesh, 60 Å pore diameter silica gel. ¹H and ¹³C NMR spectra were recorded at room temperature on a Bruker AV800, DRX-500 and ARX-400. Chemical shifts (δ values) are reported in parts per million, and are referenced to the deuterated residual solvent peak. NMR data is reported as: δ value (chemical shift, J-value (Hz), integration, where s=singlet, d=doublet, t=triplet, q=quartet, brs=broad singlet). Optical rotations were recorded on a Perkin Elmer 341 polarimeter. HRMS and LRMS spectra were recorded at the Purdue University Department of Chemistry Mass Spectrometry Center. HPLC analysis and purification was done an on Agilent 1100 series instrument using a YMC Pack ODS-A column of 4.6 mm ID for analysis and either 10 mm ID or 20 mm ID for purification. The purity of all test compounds was determined by HPLC analysis to be >95% pure.

### cis-Cyclohex-4-ene-1,2-diyldimethanol (9)

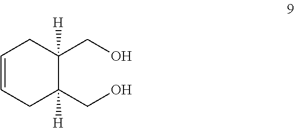

To a slurry of lithium aluminum hydride in THE was added cis-4-Cyclohexene-1,2-dicarboxylic anhydride 10 (7 g, 46.00 mmol) in THE (300 mL) at 0° C. over 20 min. The reaction mixture was stirred at 0° C. for 3 h and was quenched by dropwise addition of methanol over a period of 30 min at 0° C. The reaction mixture was allowed to warm to room temperature and added aq solution of sodium sulfate and stirred at 23° C. overnight. The resulting slurry was filtered, and the solid was rinsed with ethyl acetate. The layers were separated, and the aqueous layer was extracted with ethyl acetate. The combined organic layers were dried over Na₂SO₄, filtered and the solvent was removed under reduced pressure to give cis-diol 9 (g, 85%) as a liquid. ¹H NMR (400 MHz, CDCl₃) δ 5.62-5.59 (m, 2H), 3.71 (dd, J=11.0, 6.5 Hz, 2H), 3.58 (dd, J=11.1, 3.3 Hz, 2H), 3.27 (brs, 2H), 2.18-1.96 (m, 6H); ¹³C NMR (101 MHz, CDCl₃) δ 125.4, 64.0, 37.7, 26.8. LRMS-ESI (m/z): 143.0 [M+H]+. Spectral data was identical with the reported data¹.

### ((1S,6R)-6-(Hydroxymethyl)cyclohex-3-en-1-yl)methyl acetate (11)

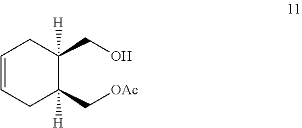 [SMILES: CC(=O)OC[C@H]1CC=CC[C@H]1CO]

A mixture of diol 9 (500 mg, 3.52 mmol) and PPL (porcine pancreatic lipase Sigma type 2, 2.27 g) in ethyl acetate (50 mL) were stirred at 23° C. for 12 h. After completion of diol by TLC, the reaction mixture was filtered and the solvent was removed in vacuo to give a crude residue which was purified by column chromatography over silica gel (30% EtOAc/hexanes) to afford the mono acetate 11 (530 mg, 82%) along with di acetate (40 mg, 5%) as minor product. R_{f}=0.5 (50% EtOAc/hexanes). [α]_{D}²⁰ +17.5 (c 2.2, CHCl₃), {literature data²: [α]_{D}²⁰ +19.0 (c 5.85, CHCl₃)}; ¹H NMR (400 MHz, CDCl₃) δ 5.67-5.57 (m, 2H), 4.17 (dd, J=11.0, 6.0 Hz, 1H), 3.94 (dd, J=11.0, 8.0 Hz, 1H), 3.68-3.53 (m, 2H), 2.27-2.20 (m, 1H), 2.17-2.05 (m, 3H), 2.04 (s, 3H), 2.03-1.82 (m, 3H). ¹³C NMR (101 MHz, CDCl₃) δ 171.3, 125.5, 124.99, 64.9, 63.5, 37.1, 33.1, 26.9, 25.9, 20.9. LRMS-ESI (m/z): 185.1 [M+H]+. Spectral data was identical with the reported data¹.

References: (1) J. Am. Chem. Soc. 2012, 134, 4037-4040. (2) J. Org. Chem. 1995, 60, 2506-2513.

### ((1S,6R)-6-(Dimethoxymethyl)cyclohex-3-en-1-yl)methyl acetate (12)

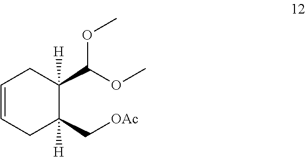 [SMILES: COC(OC)[C@@H]1CC=CC[C@@H]1COC(C)=O]

Oxalyl chloride (1.34 mL, 15.21 mmol) in dry CH₂Cl₂ (60 mL) was cooled to −78° C. under nitrogen atmosphere. Dimethyl sulfoxide (2.2 mL, 30.43 mmol) was added dropwise. After 15 min, alcohol 11 (1.4 g, 7.60 mmol) in dry CH₂Cl₂ (20 mL) was added to the reaction mixture via cannula and stirred for 30 min at −78° C. Then Et₃N (5.3 mL, 38.04 mmol) was added and the mixture stirred for 15 min. Further reaction was carried out at 0° C. The solvent was concentrated, extracted with EtOAc (2×100 mL), and washed with H₂O and brine, and the organic layer was dried over Na₂SO₄, filtered, and concentrated under reduced pressure. The residue was purified by flash column chromatography (15% EtOAc/hexanes) to afford aldehyde (1.25 g, 90%) as a colorless oil. R_{f}=0.5 (30% EtOAc/hexanes). [α]_{D}²⁰ −47.9 (c 0.73, CHCl₃); ¹H NMR (400 MHz, CDCl₃) δ 9.71 (s, 1H), 5.73-5.61 (m, 2H), 4.07 (ddq, J=6.6, 3.4, 0.9 Hz, 2H), 2.65-2.55 (m, 2H), 2.33-2.24 (m, 3H), 2.07-1.96 (m, 4H); ¹³C NMR (100 MHz, CDCl₃) δ 203.24, 170.63, 125.34, 124.70, 64.38, 47.18, 32.68, 26.77, 22.62, 20.65. LRMS-ESI (m/z): 183.0 [M+H]⁺.

To a stirred solution of above aldehyde (1.25 g, 6.86 mmol) in methanol (20 mL) were added trimethyl orthoformate (7.5 mL, 68.60 mmol) followed by tetrabutylammonium bromide (66 mg, 0.137 mmol) at 23° C. The reaction mixture was stirred for 8 h at 23° C. After this period, the reaction mixture was quenched by the addition of saturated aqueous NH₄Cl solution. Methanol was removed under reduced pressure and the reaction mixture was diluted with ethyl acetate. The layers were separated and the aqueous layer was extracted with EtOAc, and combined organic extracts were dried over Na₂SO₄ and concentrated under reduced pressure. The crude product was purified by silica gel column chromatography (10% EtOAc/hexanes) to afford 12 (1.32 g, 84%). R_{f}=0.5 (10% EtOAc/hexanes, 3 times). [α]_{D}²⁰+5.9 (c 1.2, CHCl₃). ¹H NMR (400 MHz, CDCl₃) δ 5.70-5.54 (m, 2H), 4.33 (dd, J=8.3, 1.9 Hz, 1H), 4.12 (ddd, J=10.8, 5.4, 1.8 Hz, 1H), 3.99 (ddd, J=10.8, 9.0, 1.9 Hz, 1H), 3.36-3.30 (m, 6H), 2.29 (m, 1H), 2.21-1.99 (m, 7H), 1.88 (dddd, J=16.1, 7.9, 4.3, 2.2 Hz, 1H); ¹³C NMR (100 MHz, CDCl₃) δ 171.09, 125.72, 124.75, 104.98, 64.26, 53.48, 52.51, 37.41, 31.89, 27.52, 24.49, 20.98. LRMS-ESI (m/z): 251.1 [M+Na]⁺.

### (1R,2S,4R,5S)-4-(Dimethoxymethyl)-5-(hydroxymethyl)cyclohexane-1,2-diol (13) and (1S,2R,4R,5S)-4-(Dimethoxymethyl)-5-(hydroxymethyl)cyclohexane-1,2-diol (14)

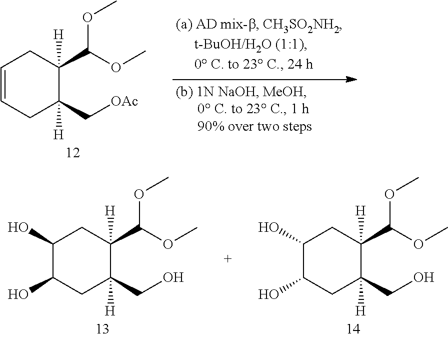

AD-mix-β (3.0 g) was dissolved in 1:1 tert-butyl alcohol/water (22 mL) and the mixture was stirred for 10 min. MeSO₂NH₂ (208 mg, 2.19 mmol) was then added, and stirring was continued for a further 10 min. After the mixture was cooled to 0° C., 12 (500 mg, 2.19 mmol) in t-BuOH (2 mL) was added. The reaction was slowly warmed to ambient temperature and stirred for 24 h. At this time solid Na₂SO₃ was added, and the reaction was stirred for an additional 30 min. The reaction was then partitioned between EtOAc/water and the aqueous layer extracted with EtOAc. The combined organic layers were washed brine solution, dried over Na₂SO₄, filtered, and concentrated under reduced pressure to yield inseparable mixture of diols which were used for the next step with further purification.

To a stirred solution of above diol was dissolved in methanol (6 mL) and added 1N NaOH (0.6 mL) at 0° C. The reaction mixture was slowly warmed to ambient temperature and stirred for 3 h. After completion of starting material, methanol was evaporated and extracted with dichloromethane (3×30 mL). The combined organic layers were dried over Na₂SO₄, filtered, and concentrated under reduced pressure. The crude residue was purified by column chromatography (5% MeOH/CH₂Cl₂) over silica gel to afford triol 13 (207 mg, 43%) and 14 (227 mg, 47%) as an oily liquids.

### Compound 13:

R_{f}=0.4 (10% MeOH/CH₂Cl₂). [α]_{D}²⁰ −3.2 (c 0.58, CHCl₃). ¹H NMR (400 MHz, Methanol-d₄) δ 4.46 (d, J=8.3 Hz, 1H), 3.78 (dt, J=16.6, 4.6 Hz, 2H), 3.58 (ddd, J=14.4, 10.9, 4.3 Hz, 2H), 3.36 (s, 3H), 3.35 (s, 3H), 2.00-1.82 (m, 3H), 1.81-1.69 (m, 2H), 1.62 (dt, J=12.9, 4.3 Hz, 1H); ¹³C NMR (100 MHz, MeOD) δ 104.94, 70.73, 68.36, 62.95, 52.41, 52.06, 38.99, 34.65, 33.23, 27.39. LRMS-ESI (m/z): 243.1 [M+Na]⁺.

### Compound 14:

R_{f}=0.2 (10% MeOH/CH₂Cl₂). [α]_{D}²⁰ −0.77 (c 3.49, CHCl₃). ¹H NMR (400 MHz, Methanol-d₄) δ 4.30 (d, J=7.8 Hz, 1H), 3.89 (dt, J=6.0, 3.0 Hz, 1H), 3.77 (dt, J=10.2, 3.7 Hz, 1H), 3.63 (dd, J=10.9, 6.0 Hz, 1H), 3.50 (dd, J=11.0, 8.8 Hz, 1H), 3.33 (s, 3H), 3.31 (s, 3H), 2.26 (ddt, J=12.0, 8.3, 4.2 Hz, 1H), 2.15-2.07 (m, 1H), 1.87-1.79 (m, 1H), 1.78-1.65 (m, 2H), 1.63-1.51 (m, 1H). ¹³C NMR (100 MHz, MeOD) δ 105.21, 68.41, 67.30, 60.32, 52.68, 51.69, 35.82, 34.33, 28.83, 28.48. LRMS-ESI (m/z): 243.1 [M+Na]⁺.

### (1R,3aS,5S,7aR)-Octahydro-1,6-epoxyisobenzofuran-5-ol (6)

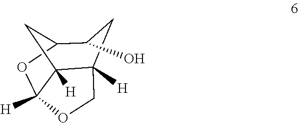

To a stirred solution of triol 13 (340 mg, 1.54 mmol) in dichloromethane (16 mL) was added 10-camphorsulfonylchloride (36 mg, 0.15 mmol) at 0° C. for 1 h. The crude residue was purified by silica gel column chromatography (40% EtOAc/hexanes) to afford alcohol 6 (198 mg, 82%) as a white amorphous solid. R_{f}=0.3 (70% EtOAc/hexanes). [α]_{D}²⁰ +45.5 (c 0.77, CHCl₃). ¹H NMR (400 MHz, CDCl₃) δ 5.64 (d, J=4.2 Hz, 1H), 4.30 (t, J=5.7 Hz, 1H), 4.03-3.87 (m, 3H), 3.21 (d, J=10.3 Hz, 1H), 2.82 (dt, J=8.8, 4.4 Hz, 1H), 2.56-2.48 (m, 1H), 1.93 (dt, J=12.4, 5.0 Hz, 1H), 1.83-1.73 (m, 2H), 1.56 (dt, J=15.4, 1.1 Hz, 1H); ¹³C NMR (100 MHz, CDCl₃) δ 108.63, 76.83, 76.60, 67.49, 42.11, 34.75, 31.79, 29.57. LRMS-ESI (m/z): 157 [M+H]⁺.

### cis-Cyclohex-4-ene-1,2-diylbis(methylene) diacetate (15)

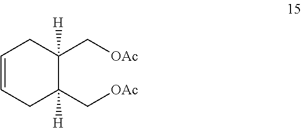 [SMILES: CC(=O)OC[C@H]1CC=CC[C@H]1COC(C)=O]

To a stirred solution of diol 9 (11.5 g, 80.98 mmol) were added pyridine (26.1 mL, 323.94 mmol), acetic anhydride (15.3 mL, 161.97 mmol) and followed by DMAP (495 mg, 4.05 mmol) at 0° C. The resulting mixture was stirred at 23° C. for overnight. Upon completion, the reaction mixture was quenched with water and extracted with EtOAc (2×100 mL). The combined organic layers were dried over NaSO4, filtered and concentrated under reduced pressure. The crude residue was purified by silica gel column chromatography (20% EtOAc/hexanes) to afford alcohol 15 (18.1 g, 98%). R_{f}=0.8 (50% EtOAc/hexanes). ¹H NMR (400 MHz, Chloroform-d) δ 5.63-5.60 (m, 2H), 4.10 (dd, J=11.0, 6.5 Hz, 2H), 4.01 (dd, J=11.0, 7.4 Hz, 2H), 2.28-2.11 (m, 4H), 2.05 (s, 6H), 1.97-1.89 (m, 2H).¹³C NMR (101 MHz, CDCl₃) δ 170.95, 125.01, 64.96, 33.57, 26.46, 20.88. LRMS-ESI (m/z): 227.1 [M+H]⁺.

### ((1R,6S)-6-(Hydroxymethyl)cyclohex-3-en-1-yl)methyl acetate (16)

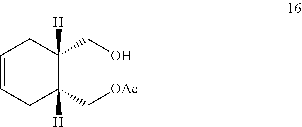 [SMILES: CC(=O)OC[C@@H]1CC=CC[C@@H]1CO]

To a stirred solution of diacetate 15 (18.6 g, 82.30 mmol) in 0.1 M Phosphate buffer (242 mL, pH 7) was added PPL (1.86 g, Sigma type II, crude) at 23° C. 1N NaHCO₃ solution (93 mL) was added dropwise and the heterogeneous mixture was stirred for 16 h. The mixture was then filtered through a pad of celite. The filtrate was extracted with dichloromethane (×3). The combined organic layers were washed with brine, dried over Na₂SO₄, filtered and the solvent was evaporated in vacuo. The residue was purified by chromatography over silica gel (30% EtOAc/hexanes) to obtain 16 (12.75 g, 84%) as a colorless oil. R_{f}=0.6 (50% EtOAc/hexanes). [α]=−17.5 (c=1.43, CHCl₃); {literature data³: [α]_{D}²³=−17.0 (c=0.42, CHCl₃)}.

Reference: (3) Tetrahedron: Asymmetry 1997, 28, 677-681.

### ((1R,6S)-6-(Dimethoxymethyl)cyclohex-3-en-1-yl)methyl acetate (17)

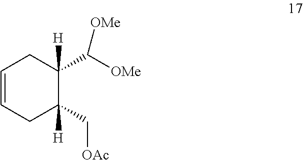 [SMILES: COC(OC)[C@H]1CC=CC[C@H]1COC(C)=O]

The title compound 17 (9 g, 80% over two steps) was obtained from 16 (9 g, 48.91 mmol) by following the procedure outlined for compound 12. R_{f}=0.5 (10% EtOAc/hexanes, 3 times). [α]_{D}²⁰ −5.5 (c 1.0, CHCl₃). ¹H and ¹³C NMR spectral data was identical with 12.

### (1S,2R,4S,5R)-4-(Dimethoxymethyl)-5-(hydroxymethyl)cyclohexane-1,2-diol (18) and (1R,2S,4S,5R)-4-(Dimethoxymethyl)-5-(hydroxymethyl)cyclohexane-1,2-diol (19)

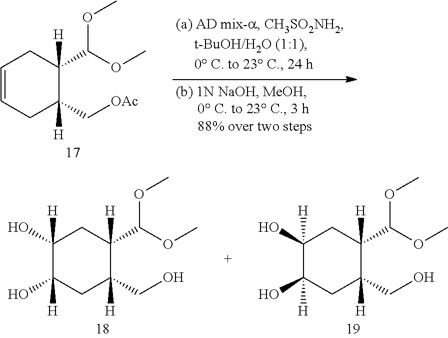

### 4-nitrophenyl ((1R,3aS,7aR)-octahydro-1,6-epoxyisobenzofuran-5-yl) carbonate

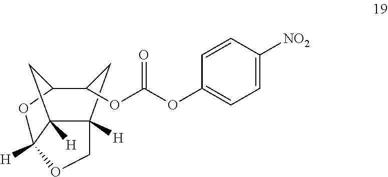

Triol 18 (405 mg, 42%) and 19 (445 mg, 46%) were synthesized from 17 (1 g, 4.38 mmol) by following the procedure outlined for compound 13 and 14.

### (1S,3aR,7aS)-Octahydro-1,6-epoxyisobenzofuran-5-ol (ent-6)

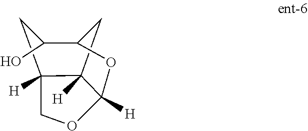

The title compound ent-6 (62 mg, 79%) was obtained from 18 (110 mg, 0.5 mmol) by following the procedure outlined for compound 6. R_{f}=0.3 (70% EtOAc/hexanes); [α]_{D}²⁰=−47.3 (c 0.76, CHCl₃). ¹H and ¹³C NMR data is identical with 6.

### 4-Nitrophenyl ((1R,3aS,7aR)-octahydro-1,6-epoxyisobenzofuran-5-yl) carbonate (19)

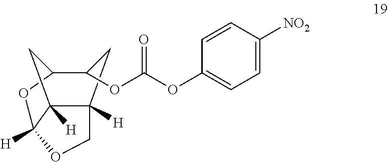

To a stirred solution of 6 (22 mg, 0.14 mmol) in dichloromethane (1.0 mL) were added pyridine (30 μL, 0.32 mmol) and 4-nitrophenylchloroformate (63 mg, 0.31 mmol) at 0° C. under argon atmosphere. The reaction mixture was warmed to 23° C. and stirred for 12 h. Upon completion, solvent was removed under reduced pressure. The crude product was purified by silica gel column chromatography (35% EtOAc in hexane) to afford 19 (39.5 mg, 87%) as an amorphous solid. R_{f}=0.5 (70% EtOAc/hexanes). [α]_{D}²⁰+77.4 (c 0.7, CHCl₃). ¹H NMR (500 MHz, CDCl₃) δ 8.27-8.23 (m, 2H), 7.43-7.38 (m, 2H), 5.69 (d, J=3.6 Hz, 1H), 4.91 (ddt, J=7.4, 5.0, 1.0 Hz, 1H), 4.62 (t, J=5.4 Hz, 1H), 4.18 (t, J=8.3 Hz, 1H), 3.93 (dd, J=8.8, 2.9 Hz, 1H), 2.82 (dt, J=8.6, 3.9 Hz, 1H), 2.68-2.59 (m, 1H), 2.11-2.01 (m, 2H), 1.80 (d, J=12.7 Hz, 1H), 1.71 (d, J=14.7 Hz, 1H); ¹³C NMR (125 MHz, CDCl₃) δ 155.66, 151.70, 145.26, 125.19, 121.85, 109.05, 77.21, 76.20, 73.95, 42.32, 34.04, 29.22, 28.54. LRMS-ESI (m/z): 344 [M+Na]⁺.

### 4-Nitrophenyl ((1S,3aR,7aS)-octahydro-1,6-epoxyisobenzofuran-5-yl) carbonate (20)

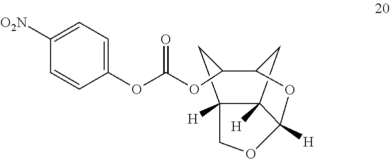

The title compound 20 (100 mg, 88%) was obtained from ent-6 (55 mg, 0.352 mmol) by following the procedure outlined for compound 19. R_{f}=0.5 (70% EtOAc/hexanes). [α]_{D}²⁰ −79.1 (c 0.8, CHCl₃). ¹H and ¹³C NMR data is identical with 19.

### (1R,3aS,7aR)-Octahydro-1,6-epoxyisobenzofuran-5-yl ((2S,3R)-3-hydroxy-4-((N-isobutyl-4-methoxyphenyl)sulfonamido)-1-phenylbutan-2-yl)carbamate (4a)

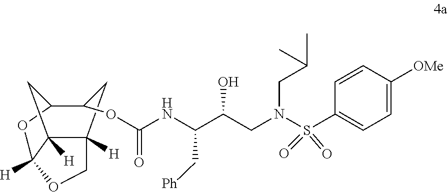

To a stirred solution of activated alcohol 19 (15 mg, 0.046 mmol) and Isostere 21 (21 mg, 0.051 mmol) in acetonitrile (2 mL) was added DIPEA (40 μL, 0.233 mmol) at 23° C. under argon atmosphere. The reaction mixture was stirred at 23° C. until completion. Upon completion, solvents were removed under reduced pressure and crude product was purified by silica gel column chromatography (50% EtOAc in hexane) to give 4a (22 mg, 81%) as an amorphous solid. R_{f}=0.3 (70% EtOAc/hexanes). ¹H NMR (500 MHz, CDCl₃) δ 7.72 (d, J=8.9 Hz, 2H), 7.30-7.23 (m, 4H), 7.22-7.17 (m, 1H), 6.97 (d, J=8.9 Hz, 1H), 5.60 (d, J=3.6 Hz, 1H), 5.02 (d, J=9.3 Hz, 1H), 4.71 (t, J=6.2 Hz, 1H), 4.31 (t, J=5.4 Hz, 1H), 4.10 (m, 1H), 3.87 (m, 1H), 3.86 (s, 3H), 3.82-3.71 (m, 3H), 3.11 (dtd, J=18.2, 15.1, 5.8 Hz, 3H), 2.97 (dd, J=13.4, 8.3 Hz, 1H), 2.80 (dd, J=13.3, 6.6 Hz, 2H), 2.72 (dt, J=8.7, 3.9 Hz, 1H), 2.50 (m, 1H), 1.96 (ddd, J=12.5, 6.0, 4.2 Hz, 1H), 1.91-1.79 (m, 3H), 1.70 (d, J=12.5 Hz, 1H), 1.28 (m, 1H), 0.91 (d, J=6.6 Hz, 3H), 0.86 (d, J=6.6 Hz, 3H); ¹³C NMR (125 MHz, CDCl₃) δ 162.83, 155.83, 137.69, 129.94, 129.45, 129.40, 128.30, 126.26, 114.19, 108.75, 76.25, 75.34, 72.37, 58.58, 55.49, 55.11, 53.54, 42.45, 36.03, 34.07, 29.29, 29.04, 27.14, 20.04, 19.76. LRMS-ESI (m/z): 589.2 [M+H]⁺; HRMS-ESI (m/z): [M+H]⁺ calcd for C₃₀H₄₁N₂O₈S, 589.2578; found 589.2572.

### (1R,3aS,7aR)-Octahydro-1,6-epoxyisobenzofuran-5-yl ((2S,3R)-4-((2-(cyclopropylamino)-N-isobutylbenzo[d]thiazole)-6-sulfonamido)-3-hydroxy-1-phenylbutan-2-yl)carbamate (4b)

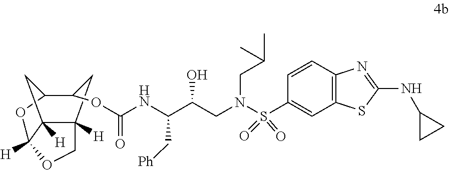

Activated alcohol 19 (8 mg, 0.024 mmol) was treated with Isostere amine 22 (14 mg, 0.027 mmol) by following the procedure outlined for inhibitor 4a to give inhibitor 4b (12.5 mg, 75%) as an amorphous solid. R_{f}=0.15 (80% EtOAc/hexanes). ¹H NMR (500 MHz, CDCl₃) δ 8.09 (s, 1H), 7.69 (d, J=8.6 Hz, 1H), 7.55 (d, J=8.5 Hz, 1H), 7.39-7.15 (m, 5H), 5.59 (d, J=3.6 Hz, 1H), 5.09 (d, J=9.7 Hz, 1H), 4.70 (m, 1H), 4.31 (t, J=5.5 Hz, 1H), 4.09 (t, J=8.2 Hz, 1H), 3.94-3.71 (m, 4H), 3.21 (dd, J=15.1, 8.9 Hz, 1H), 3.16-3.07 (m, 2H), 3.02 (dd, J=13.4, 8.5 Hz, 1H), 2.86-2.68 (m, 4H), 2.50 (m, 1H), 1.99-1.80 (m, 4H), 1.69 (d, J=12.6 Hz, 1H), 1.26 (m, 1H), 0.97-0.90 (m, 5H), 0.87 (d, J=6.5 Hz, 3H), 0.81-0.77 (m, 2H); ¹³C NMR (125 MHz, CDCl₃) δ 172.94, 155.85, 155.46, 137.72, 131.06, 130.45, 129.45, 128.32, 126.29, 125.33, 120.85, 118.41, 108.75, 76.25, 75.34, 72.38, 58.69, 55.14, 53.60, 42.44, 36.01, 34.04, 29.60, 29.28, 29.03, 27.19, 26.55, 20.07, 19.78, 7.86. LRMS-ESI (m/z): 671.2 [M+H]⁺; HRMS-ESI (m/z): [M+H]⁺ calcd for C₃₃H₄₃N₄O₇S₂, 671.2568; found 671.2574.

### (1R,3aS,7aR)-Octahydro-1,6-epoxyisobenzofuran-5-yl ((2S,3R)-3-hydroxy-4-((N-isobutyl-2-(isopropylamino)benzo[d]oxazole)-6-sulfonamido)-1-phenylbutan-2-yl)carbamate (4c)

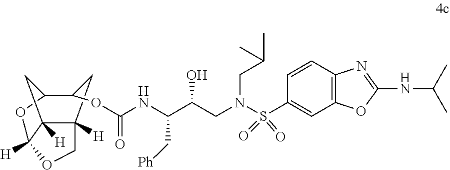

Activated alcohol 19 (10 mg, 0.031 mmol) was treated with Isostere amine 26 (16 mg, 0.034 mmol) by following the procedure outlined for inhibitor 4a to give inhibitor 4c (17.5 mg, 86%) as an amorphous solid. R_{f}=0.4 (5% MeOH/CH₂Cl₂). ¹H NMR (800 MHz, CDCl₃) δ 7.71 (d, J=1.8 Hz, 1H), 7.64 (dd, J=8.2, 1.8 Hz, 1H), 7.41 (d, J=8.3 Hz, 1H), 7.31-7.25 (m, 5H), 7.23-7.19 (m, 1H), 5.61 (d, J=3.6 Hz, 1H), 5.44 (brs, 1H), 5.09 (d, J=9.3 Hz, 1H), 4.72 (t, J=6.3 Hz, 1H), 4.33 (t, J=5.4 Hz, 1H), 4.16-4.09 (m, 2H), 3.91-3.74 (m, 4H), 3.20 (dd, J=15.1, 8.8 Hz, 1H), 3.15-3.07 (m, 2H), 3.01 (dd, J=13.4, 8.4 Hz, 1H), 2.85-2.79 (m, 2H), 2.74 (dd, J=8.8, 4.3 Hz, 1H), 2.52 (m, 1H), 1.97 (dt, J=12.6, 4.7 Hz, 1H), 1.90-1.85 (m, 2H), 1.81 (brs, 1H), 1.72 (d, J=12.6 Hz, 1H), 1.38 (d, J=6.5 Hz, 6H), 1.31-1.27 (m, 1H), 0.94 (d, J=6.6 Hz, 3H), 0.89 (d, J=6.6 Hz, 3H); ¹³C NMR (200 MHz, CDCl₃) δ 163.31, 155.95, 147.92, 147.67, 137.83, 130.05, 129.55, 128.43, 126.40, 124.23, 115.97, 108.86, 108.46, 76.37, 75.44, 72.45, 58.78, 55.25, 53.71, 45.75, 42.56, 36.11, 34.18, 29.39, 29.13, 27.28, 22.97, 20.17, 19.89. LRMS-ESI (m/z): 657.2 [M+H]⁺; HRMS-ESI (m/z): [M+H]⁺ calcd for C₃₃H₄₅N₄O₈S, 657.2953; found 657.2947.

### (1R,3aS,7aR)-Octahydro-1,6-epoxyisobenzofuran-5-yl ((2S,3R)-4-((2-(cyclopropylamino)-N-isobutylbenzo[d]thiazole)-6-sulfonamido)-1-(3,5-difluorophenyl)-3-hydroxybutan-2-yl)carbamate (4d)

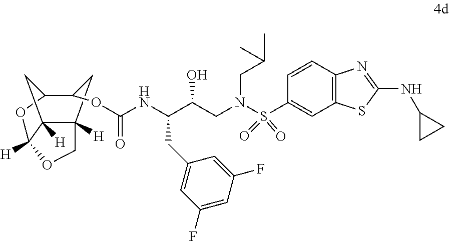

Activated alcohol 19 (7 mg, 0.021 mmol) was treated with Isostere amine 23 (13 mg, 0.023 mmol) by following the procedure outlined for inhibitor 4a to give inhibitor 4d (10 mg, 65%) as an amorphous solid. R_{f}=0.1 (70% EtOAc/hexanes). ¹H NMR (500 MHz, CDCl₃) δ 8.11 (s, 1H), 7.71 (d, J=10.0 Hz, 1H), 7.57 (d, J=8.5 Hz, 1H), 6.90 (brs, 1H), 6.80 (d, J=7.6 Hz, 2H), 6.64 (dd, J=10.1, 7.7 Hz, 1H), 5.60 (d, J=3.6 Hz, 1H), 5.13 (d, J=8.9 Hz, 1H), 4.73 (m, 1H), 4.32 (t, J=5.2 Hz, 1H), 4.13 (t, J=8.1 Hz, 1H), 3.96-3.76 (m, 3H), 3.22-3.07 (m, 3H), 3.02 (dd, J=13.4, 8.4 Hz, 1H), 2.85 (dd, J=13.4, 6.8 Hz, 1H), 2.81-2.70 (m, 3H), 2.53 (m, 1H), 2.03-1.80 (m, 4H), 1.71 (d, J=12.6 Hz, 1H), 1.34 (d, J=16.0 Hz, 1H), 0.97-0.91 (m, 5H), 0.89 (d, J=6.5 Hz, 3H), 0.82-0.76 (m, 2H); ¹³C NMR (200 MHz, CDCl₃) δ 172.82, 163.51 (d), 162.28 (d), 155.84, 155.58, 142.16, 131.29, 130.57, 125.43, 120.97, 118.75, 112.40 (d), 108.89, 101.95 (t), 76.43, 75.34, 72.63, 72.44, 58.96, 55.02, 53.72, 42.54, 35.89, 34.22, 29.71, 29.39, 29.13, 27.37, 26.72, 20.17, 19.89, 8.04. LRMS-ESI (m/z): 707.2 [M+H]⁺; HRMS-ESI (m/z): [M+H]⁺ calcd for C₃₃H₄₁F₂N₄O₇S₂, 707.2379; found 707.2385.

### (1R,3aS,7aR)-Octahydro-1,6-epoxyisobenzofuran-5-yl ((2S,3R)-4-((2-(cyclopropylamino)-N-isobutylbenzo[d]thiazole)-6-sulfonamido)-1-(3-fluorophenyl)-3-hydroxybutan-2-yl)carbamate (4e)

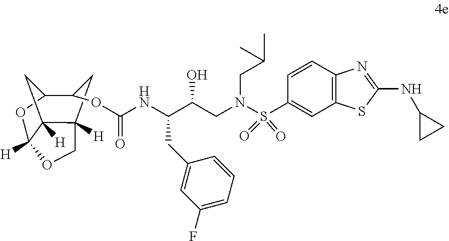

Activated alcohol 19 (12 mg, 0.037 mmol) was treated with Isostere amine 24 (21 mg, 0.041 mmol) by following the procedure outlined for inhibitor 4a to give inhibitor 4e (18 mg, 70%) as an amorphous solid. R_{f}=0.2 (80% EtOAc/hexanes). ¹H NMR (500 MHz, CDCl₃) δ 8.13 (d, J=1.9 Hz, 1H), 7.74 (dd, J=8.4, 2.0 Hz, 1H), 7.59 (d, J=8.9 Hz, 1H), 7.26 (dd, J=8.1, 6.2 Hz, 1H), 7.18 (brs, 1H), 7.07 (d, J=7.7 Hz, 1H), 7.00 (m, 1H), 6.92 (t, J=8.6 Hz, 1H), 5.63 (d, J=3.7 Hz, 1H), 5.10 (d, J=9.2 Hz, 1H), 4.75 (m, 1H), 4.35 (t, J=5.6 Hz, 1H), 4.14 (t, J=8.3 Hz, 1H), 3.94-3.77 (m, 4H), 3.23 (dd, J=15.2, 8.4 Hz, 1H), 3.20-3.07 (m, 2H), 3.05 (dd, J=13.4, 8.3 Hz, 1H), 2.91-2.71 (m, 4H), 2.54 (m, 1H), 2.07-1.85 (m, 4H), 1.73 (d, J=12.8 Hz, 1H), 1.34 (d, J=10.0 Hz, 1H), 1.00-0.88 (m, 8H), 0.84-0.80 (m, 2H); ¹³C NMR (200 MHz, CDCl₃) δ 172.88, 163.42 (d), 162.20 (d), 155.90, 155.51, 140.59 (d), 131.22, 130.67, 129.83 (d), 125.36 (d), 120.98, 118.67, 116.42 (d), 113.32 (d), 108.88, 76.39, 75.39, 72.55, 72.46, 58.86, 55.15, 53.71, 42.55, 35.87, 34.21, 31.93, 29.71, 29.49, 29.40, 29.37, 29.13, 27.34, 26.71, 20.17, 19.90, 8.02. LRMS-ESI (m/z): 689.2 [M+H]⁺; HRMS-ESI (m/z): [M+H]⁺ calcd for C₃₃H₄₂FN₄O₇S₂, 689.2474; found 689.2466.

### (1R,3aS,7aR)-Octahydro-1,6-epoxyisobenzofuran-5-yl ((2S,3R)-4-((2-(cyclopropylamino)-N-isobutylbenzo[d]thiazole)-6-sulfonamido)-1-(4-fluorophenyl)-3-hydroxybutan-2-yl)carbamate (4f)

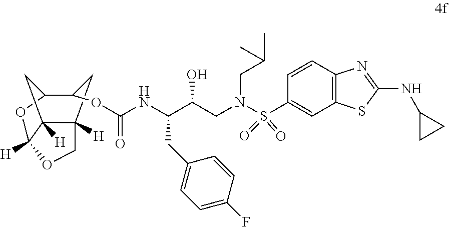

Activated alcohol 19 (12 mg, 0.037 mmol) was treated with Isostere amine 25 (21 mg, 0.041 mmol) by following the procedure outlined for inhibitor 4a to give inhibitor 4f (17.5 mg, 68%) as an amorphous solid. R_{f}=0.2 (80% EtOAc/hexanes). ¹H NMR (500 MHz, CDCl₃) δ 8.10 (d, J=1.9 Hz, 1H), 7.70 (dd, J=8.5, 1.9 Hz, 1H), 7.57 (d, J=8.5 Hz, 1H), 7.22 (dd, J=8.2, 5.3 Hz, 2H), 6.97 (t, J=8.7 Hz, 2H), 6.89 (brs, 1H), 5.60 (d, J=3.6 Hz, 1H), 5.02 (d, J=9.0 Hz, 1H), 4.70 (t, J=6.1 Hz, 1H), 4.33 (t, J=5.4 Hz, 1H), 4.11 (t, J=8.2 Hz, 1H), 3.92-3.71 (m, 4H), 3.18 (dd, J=15.1, 8.2 Hz, 1H), 3.14-3.06 (m, 2H), 3.01 (dd, J=13.4, 8.5 Hz, 1H), 2.83 (dd, J=13.4, 6.7 Hz, 1H), 2.79-2.70 (m, 3H), 2.52 (m, 1H), 1.96 (dt, J=12.5, 5.2 Hz, 1H), 1.92-1.81 (m, 2H), 1.70 (d, J=12.6 Hz, 1H), 1.29 (d, J=10.0 Hz, 1H), 0.98-0.90 (m, 5H), 0.88 (d, J=6.6 Hz, 3H), 0.82-0.77 (m, 2H); ¹³C NMR (200 MHz, CDCl₃) δ 172.88, 162.25, 161.03, 155.98, 133.58, 131.02, 130.66, 125.43, 120.97, 118.69, 115.24, 115.13, 108.88, 76.38, 75.38, 72.59, 72.48, 58.86, 55.29, 53.72, 42.56, 35.20, 34.20, 29.71, 29.39, 29.14, 27.34, 26.70, 20.17, 19.91, 8.02. LRMS-ESI (m/z): 689.2 [M+H]⁺; HRMS-ESI (m/z): [M+H]⁺ calcd for C₃₃H₄₂FN₄O₇S₂, 689.2474; found 689.2481.

### (1S,3aR,5S,7aS)-Octahydro-1,6-epoxyisobenzofuran-5-yl ((2S,3R)-3-hydroxy-4-((N-isobutyl-4-methoxyphenyl)sulfonamido)-1-phenylbutan-2-yl)carbamate (5a)

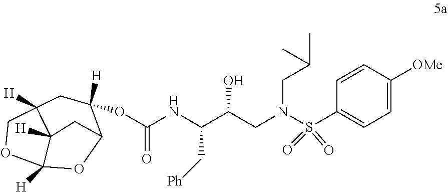

Activated alcohol 20 (15 mg, 0.046 mmol) was treated with Isostere amine 21 (21 mg, 0.051 mmol) by following the procedure outlined for inhibitor 4a to give inhibitor 5a (22 mg, 80%) as an amorphous solid. R_{f}=0.3 (70% EtOAc/hexanes). ¹H NMR (800 MHz, CDCl₃) δ 7.72 (d, J=8.4 Hz, 2H), 7.33-7.25 (m, 4H), 7.22 (t, J=7.2 Hz, 1H), 6.98 (d, J=8.4 Hz, 2H), 5.65 (s, 1H), 5.17 (d, J=8.8 Hz, 1H), 4.80-4.68 (m, 1H), 4.33 (t, J=5.4 Hz, 1H), 4.13 (m, 1H), 3.88 (s, 3H), 3.91-3.75 (m, 4H), 3.13-2.91 (m, 4H), 2.87-2.71 (m, 2H), 2.56 (m, 1H), 2.06-1.68 (m, 5H), 1.52 (d, J=15.8 Hz, 1H), 1.31 (m, 1H), 0.91 (d, J=6.6 Hz, 3H), 0.87 (d, J=6.7 Hz, 3H); ¹³C NMR (200 MHz, CDCl₃) δ 162.95, 156.18, 137.81, 130.08, 129.62, 129.49, 128.47, 126.45, 114.29, 108.87, 76.58, 75.67, 72.63, 72.14, 58.59, 55.62, 55.45, 53.53, 42.72, 35.24, 34.32, 29.70, 29.52, 29.49, 27.24, 20.15, 19.90. LRMS-ESI (m/z): 589.2 [M+H]⁺; HRMS-ESI (m/z): [M+H]⁺ calcd for C₃₀H₄₀N₂O₈SNa, 611.2398; found 611.2410.

### (1S,3aR,5S,7aS)-Octahydro-1,6-epoxyisobenzofuran-5-yl ((2S,3R)-4-((2-(cyclopropylamino)-N-isobutylbenzo[d]thiazole)-6-sulfonamido)-3-hydroxy-1-phenylbutan-2-yl)carbamate (5b)

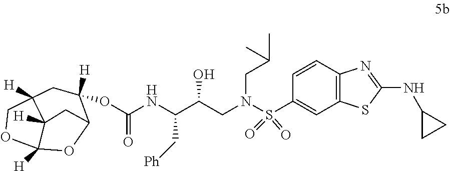

Activated alcohol 20 (15 mg, 0.046 mmol) was treated with Isostere amine 22 (25 mg, 0.051 mmol) by following the procedure outlined for inhibitor 4a to give inhibitor 5b (22 mg, 70%) as an amorphous solid. R_{f}=0.2 (80% EtOAc/hexanes). ¹H NMR (800 MHz, CDCl₃) δ 8.08 (s, 1H), 7.69 (d, J=8.5 Hz, 1H), 7.57 (d, J=8.5 Hz, 1H), 7.32-7.26 (m, 5H), 7.24-7.16 (m, 2H), 5.65 (d, J=3.6 Hz, 1H), 5.19 (d, J=8.8 Hz, 1H), 4.78 (t, J=6.1 Hz, 1H), 4.34 (t, J=5.4 Hz, 1H), 4.13 (t, J=8.3 Hz, 1H), 3.98-3.81 (m, 4H), 3.15 (dd, J=15.0, 8.7 Hz, 1H), 3.10-2.95 (m, 3H), 2.85 (dd, J=13.5, 6.9 Hz, 1H), 2.80-2.70 (m, 2H), 2.55 (m, 1H), 2.05-1.67 (m, 5H), 1.52 (d, J=15.8 Hz, 1H), 0.97 (d, J=6.5 Hz, 2H), 0.92 (d, J=6.6 Hz, 3H), 0.88 (d, J=6.6 Hz, 3H), 0.81 (m, 2H); ¹³C NMR (200 MHz, CDCl₃) δ 172.98, 156.19, 155.60, 137.81, 131.20, 130.60, 129.62, 128.49, 126.49, 125.40, 120.93, 118.55, 108.88, 76.59, 75.67, 72.67, 72.20, 58.71, 55.48, 53.63, 42.72, 35.24, 34.32, 29.71, 29.50, 27.31, 26.68, 20.18, 19.91, 7.97. LRMS-ESI (m/z): 671.2 [M+H]⁺; HRMS-ESI (m/z): [M+H]⁺ calcd for C₃₃H₄₃N₄O₇S₂, 671.2568; found 671.2563.

### (1S,3aR,5S,7aS)-Octahydro-1,6-epoxyisobenzofuran-5-yl ((2S,3R)-3-hydroxy-4-((N-isobutyl-2-(isopropylamino)benzo[d]oxazole)-6-sulfonamido)-1-phenylbutan-2-yl)carbamate (5c)

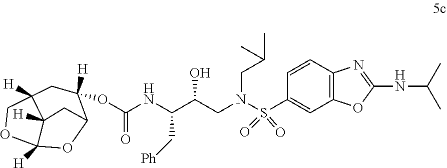

Activated alcohol 20 (8 mg, 0.024 mmol) was treated with Isostere amine 26 (13 mg, 0.027 mmol) by following the procedure outlined for inhibitor 4a to give inhibitor 5c (14 mg, 86%) as an amorphous solid. R_{f}=0.4 (5% MeOH/CH₂Cl₂). ¹H NMR (800 MHz, CDCl₃) δ 7.66 (s, 1H), 7.60 (d, J=8.2 Hz, 1H), 7.39 (d, J=8.2 Hz, 1H), 7.30-7.23 (m, 5H), 7.20 (t, J=7.1 Hz, 1H), 5.63 (d, J=3.5 Hz, 1H), 5.23 (brs, 1H), 5.07 (d, J=8.9 Hz, 1H), 4.76 (m, 1H), 4.32 (m, 1H), 4.15-4.05 (m, 2H), 3.87-3.72 (m, 4H), 3.13-2.92 (m, 4H), 2.85-2.67 (m, 2H), 2.53 (m, 1H), 2.03-1.95 (m, 2H), 1.92-1.77 (m, 2H), 1.73 (d, J=12.5 Hz, 1H), 1.50 (d, J=15.8 Hz, 1H), 1.36 (d, J=6.5 Hz, 6H), 0.90 (d, J=6.6 Hz, 3H), 0.86 (d, J=6.6 Hz, 3H); ¹³C NMR (200 MHz, CDCl₃) δ 163.27, 156.16, 147.91, 137.74, 130.09, 129.61, 128.50, 126.50, 124.21, 116.00, 108.88, 108.45, 76.60, 75.69, 72.70, 72.15, 58.75, 55.99, 55.45, 53.66, 45.79, 42.74, 35.26, 34.32, 29.70, 29.51, 27.29, 22.97, 20.17, 19.90. LRMS-ESI (m/z): 657.2 [M+H]⁺; HRMS-ESI (m/z): [M+H]⁺ calcd for C₃₃H₄₅N₄O₈S, 657.2953; found 657.2948.

### (1S,3aR,5S,7aS)-Octahydro-1,6-epoxyisobenzofuran-5-yl ((2S,3R)-4-((2-(cyclopropylamino)-N-isobutylbenzo[d]thiazole)-6-sulfonamido)-1-(3,5-difluorophenyl)-3-hydroxybutan-2-yl)carbamate (5d)

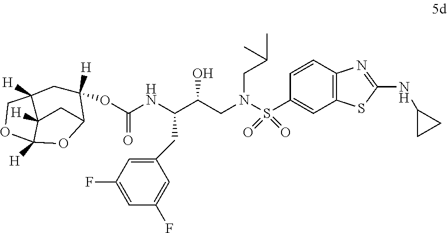

Activated alcohol 20 (12 mg, 0.037 mmol) was treated with Isostere amine 23 (21 mg, 0.041 mmol) by following the procedure outlined for inhibitor 4a to give inhibitor 5d (15.5 mg, 59%) as an amorphous solid. R_{f}=0.3 (5% MeOH/CH₂Cl₂). ¹H NMR (800 MHz, CDCl₃) δ 8.11 (s, 1H), 7.72 (d, J=8.6 Hz, 1H), 7.59 (d, J=8.5 Hz, 1H), 6.98 (brs, 1H), 6.87-6.80 (m, 2H), 6.67 (m, 1H), 5.65 (d, J=3.6 Hz, 1H), 5.25 (d, J=8.8 Hz, 1H), 4.83-4.73 (m, 1H), 4.36 (t, J=5.4 Hz, 1H), 4.16-4.05 (m, 2H), 3.95-3.73 (m, 3H), 3.18-2.93 (m, 4H), 2.89 (dd, J=13.3, 6.9 Hz, 1H), 2.84-2.72 (m, 3H), 2.56 (q, J=8.2 Hz, 1H), 2.05-1.95 (m, 2H), 1.94-1.83 (m, 2H), 1.76 (d, J=12.6 Hz, 1H), 1.72 (m, 1H), 1.52 (d, J=15.9 Hz, 1H), 0.97 (d, J=6.6 Hz, 2H), 0.94 (d, J=6.5 Hz, 3H), 0.90 (d, J=6.9 Hz, 3H), 0.83-0.79 (m, 2H). ¹³C NMR (200 MHz, CDCl₃) δ 172.96, 163.57 (d), 162.33 (d), 156.18, 155.65, 142.17, 131.29, 130.43, 125.37, 120.92, 118.67, 112.42 (d), 108.89, 102.01 (t), 76.55, 75.46, 72.75, 72.33, 58.99, 55.29, 53.67, 42.64, 34.77, 34.31, 29.70, 29.46, 27.38, 26.70, 20.16, 19.93, 8.00. LRMS-ESI (m/z): 707.2 [M+H]⁺; HRMS-ESI (m/z): [M+H]⁺ calcd for C₃₃H₄₁F₂N₄O₇S₂, 707.2379; found 707.2384.

### (1S,3aR,5S,7aS)-Octahydro-1,6-epoxyisobenzofuran-5-yl ((2S,3R)-4-((2-(cyclopropylamino)-N-isobutylbenzo[d]thiazole)-6-sulfonamido)-1-(3-fluorophenyl)-3-hydroxybutan-2-yl)carbamate (5e)

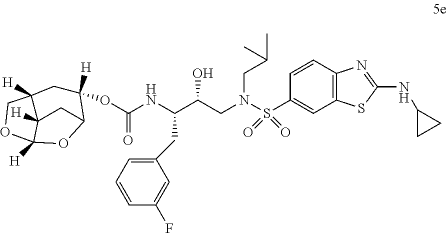

Activated alcohol 20 (4 mg, 0.012 mmol) was treated with Isostere amine 24 (7 mg, 0.013 mmol) by following the procedure outlined for inhibitor 4a to give inhibitor 5e (7.5 mg, 87%) as an amorphous solid. R_{f}=0.2 (80% EtOAc/hexanes). ¹H NMR (800 MHz, CDCl₃) δ 8.14-8.07 (m, 1H), 7.73-7.65 (m, 1H), 7.56 (dd, J=8.3, 2.8 Hz, 1H), 7.23 (m, 1H), 7.08-7.00 (m, 2H), 6.98 (d, J=10.2 Hz, 1H), 6.89 (td, J=8.8, 3.3 Hz, 1H), 5.63 (d, J=3.6 Hz, 1H), 5.21 (d, J=8.8 Hz, 1H), 4.77 (t, J=6.3 Hz, 1H), 4.32 (t, J=5.2 Hz, 1H), 4.11 (td, J=8.9, 8.5, 3.2 Hz, 1H), 4.02-3.73 (m, 4H), 3.15-2.92 (m, 4H), 2.85 (dd, J=13.3, 6.7 Hz, 1H), 2.79-2.70 (m, 2H), 2.57-2.48 (m, 1H), 2.01-1.93 (m, 2H), 1.89-1.81 (m, 2H), 1.73 (d, J=12.5 Hz, 1H), 1.50 (d, J=15.8 Hz, 1H), 0.95-0.84 (m, 8H), 0.81-0.77 (m, 2H); ¹³C NMR (200 MHz, CDCl₃) δ 172.96, 163.44, 162.22, 156.18, 155.59, 140.60, 131.23, 130.62, 130.53, 129.85 (q), 125.39 (t), 120.95 (d), 118.61, 116.47 (d), 113.37 (t), 108.88, 76.47 (d), 75.47 (d), 72.68, 72.27, 58.84, 55.39, 55.16, 53.65, 42.68, 42.55, 34.84, 34.31, 34.21, 29.70, 29.48, 29.39, 29.12, 27.34, 26.70, 20.17, 19.92, 7.99. LRMS-ESI (m/z): 689.2 [M+H]⁺; HRMS-ESI (m/z): [M+H]⁺ calcd for C₃₃H₄₂FN₄O₇S₂, 689.2474; found 689.2481.

### (1S,3aR,5S,7aS)-Octahydro-1,6-epoxyisobenzofuran-5-yl ((2S,3R)-4-((2-(cyclopropylamino)-N-isobutylbenzo[d]thiazole)-6-sulfonamido)-1-(4-fluorophenyl)-3-hydroxybutan-2-yl)carbamate (5f)

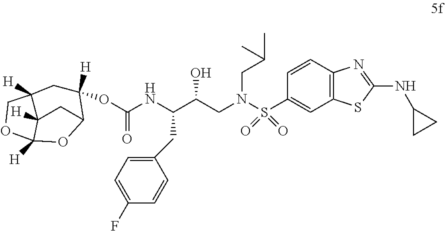

Activated alcohol 20 (4 mg, 0.012 mmol) was treated with Isostere amine 25 (7 mg, 0.013 mmol) by following the procedure outlined for inhibitor 4a to give inhibitor 5f (6.6 mg, 77%) as an amorphous solid. R_{f}=0.2 (80% EtOAc/hexanes). ¹H NMR (800 MHz, CDCl₃) δ 8.07 (s, 1H), 7.67 (d, J=8.6 Hz, 1H), 7.56 (d, J=8.5 Hz, 1H), 7.25-7.20 (m, 2H), 6.97 (t, J=8.7 Hz, 2H), 6.83 (brs, 1H), 5.63 (d, J=3.5 Hz, 1H), 5.10 (d, J=9.1 Hz, 1H), 4.74 (m, 1H), 4.32 (t, J=5.5 Hz, 1H), 4.11 (t, J=7.9 Hz, 1H), 3.90-3.72 (m, 4H), 3.12 (dd, J=15.0, 8.5 Hz, 1H), 3.07-3.03 (m, 1H), 3.01-2.96 (m, 2H), 2.91 (dd, J=14.2, 8.2 Hz, 1H), 2.83 (dd, J=13.5, 6.8 Hz, 1H), 2.77-2.69 (m, 2H), 2.53 (m, 1H), 2.03-1.95 (m, 2H), 1.89-1.80 (m, 2H), 1.73 (d, J=12.5 Hz, 1H), 1.48 (d, J=15.8 Hz, 1H), 0.96-0.93 (m, 2H), 0.91 (d, J=6.6 Hz, 3H), 0.86 (d, J=6.7 Hz, 3H), 0.81-0.78 (m, 2H); ¹³C NMR (200 MHz, CDCl3) δ 172.89, 162.30, 161.08, 156.09, 155.53, 133.39, 131.23, 131.12, 131.10, 130.58, 125.38, 120.92, 118.67, 115.30, 115.20, 108.89, 76.57, 75.53, 72.68, 72.14, 58.82, 55.37, 53.74, 42.68, 34.47, 34.32, 29.71, 29.47, 29.43, 27.33, 26.72, 20.18, 19.90, 8.02. LRMS-ESI (m/z): 689.2 [M+H]⁺; HRMS-ESI (m/z): [M+H]⁺ calcd for C₃₃H₄₂FN₄O₇S₂, 689.2474; found 689.2478.

The present invention provides for the following example embodiments, the numbering of which is not to be construed as designating levels of importance:

Embodiment 1 relates to a compound of the formula (I)

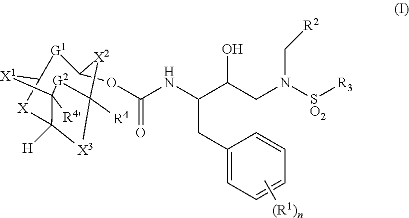

or a pharmaceutically acceptable salt, polymorph, prodrug, solvate or clathrate thereof, wherein:
n is an integer from 0 to 3; G¹ and G² are each independently (—CHR⁵—)ₚ, wherein p is 0 or 1 and each R⁵ is independently H or alkyl;
X is (—CHR⁵—)ₘO—, wherein m is 0, 1 or 2 and each R⁵ is independently H or alkyl;
X³ is (—CHR⁵—)_{d}O—, wherein d is 1 or 2 and each R⁵ is independently H or alkyl;
X¹ and X² are each, independently, (—CHR⁵—)ₘ, wherein m is 0, 1 or 2 and each R⁵ is independently H or alkyl; each R¹ is independently alkyl, alkoxy, aryl, heterocyclyl, halo, hydroxy or amino;
R² is alkyl;
R³ is aryl, benzthiazole, benzoxazole, benzofuranyl or indolyl; and
R⁴ and R^{4′} are each independently H or alkyl.

Embodiment 2 relates to the compound of Embodiment 1, wherein

X¹ is (—CHR⁵—)ₘ, with m being 2 and X² being (—CHR⁵—)ₘ, with m being 1;
X¹ and X² are each (—CHR⁵—)ₘ, with each m being 1;
X¹ is (—CHR⁵—)ₘ, with m being 0 and X² is (—CHR⁵—)ₘ with m being 1; or
X¹ and X² are each (—CHR⁵—)ₘ, with m being 0, provided that at least one of G¹ and G² is (—CHR⁵—)ₚ, wherein at least one p is 1.

Embodiment 3 relates to the compound of Embodiment 1 or 2, wherein:

### X is O;

### X³ is O; or

### X and X³ are O.

Embodiment 4 relates to the compound of Embodiments 1-3, wherein at least one p is 0, such that at least one of G¹ and G² are a bond.

Embodiment 5 relates to the compound of Embodiments 1-4, wherein R⁴ and R^{4′} are each independently H or alkyl.

Embodiment 6 relates to the compound of Embodiments 1-3, wherein:

each p is 0;
X and X³ are each O,
X¹ and X² are each independently (—CHR⁵—)ₘ; and
R⁴ and R^{4′} are each H.

Embodiment 7 relates to the compound of Embodiment 1, wherein the compound of formula (I) is a compound of formula:

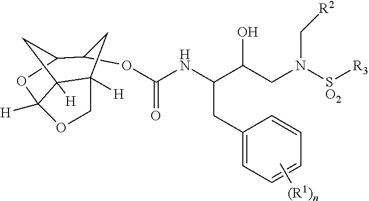

Embodiment 8 relates to the compound of Embodiment 1, wherein the compound of formula (I) is a compound of formula:

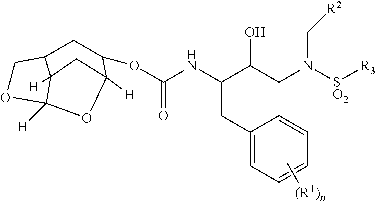

Embodiment 9 relates to the compound of Embodiments 1-8, wherein R³ is unsubstituted or substituted aryl.

Embodiment 10 relates to the compound of Embodiments 1-9, wherein R³ is selected from the group consisting of:

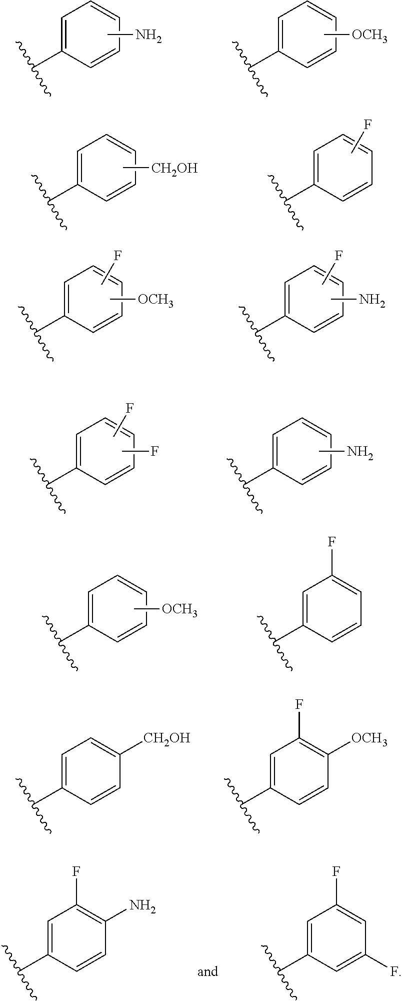

Embodiment 11 relates to the compound of Embodiment 1-8, wherein R³ is a benzthiazole or a benzoxazole:

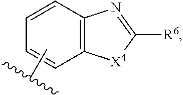

wherein R⁶ is alkyl, alkylamino, cycloalkylamino, cycloalkyl heterocycloamino, heterocyclo cycloalkylamino or heterocycloamino; and
X⁴ is S, O or NR⁷, wherein R⁷ is H, alklyl, cycloalkyl or alkylaryl. X⁴ can be S or O.

Embodiment 12 relates to the compound of Embodiments 1-8, wherein the compound is a compound of formula:

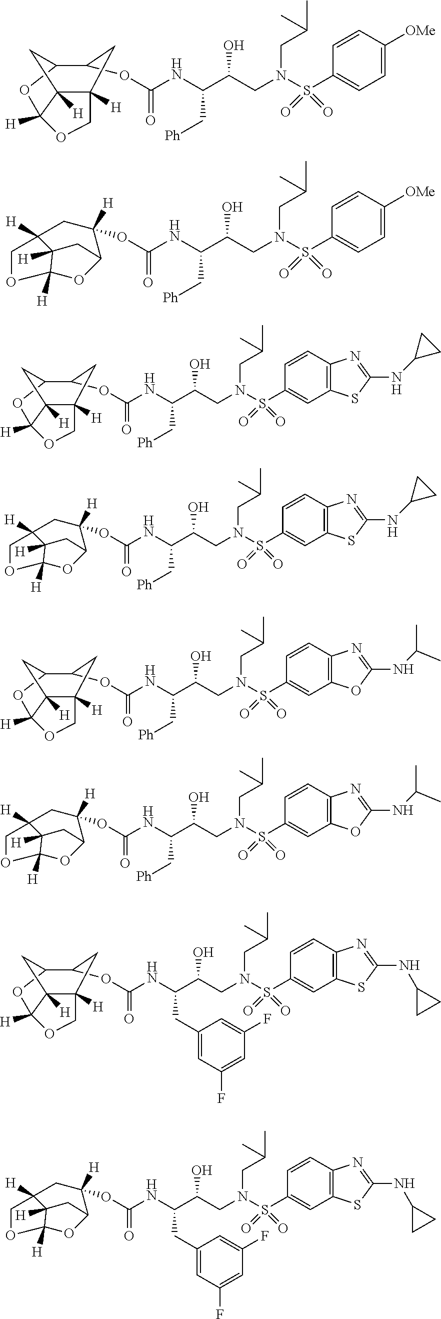

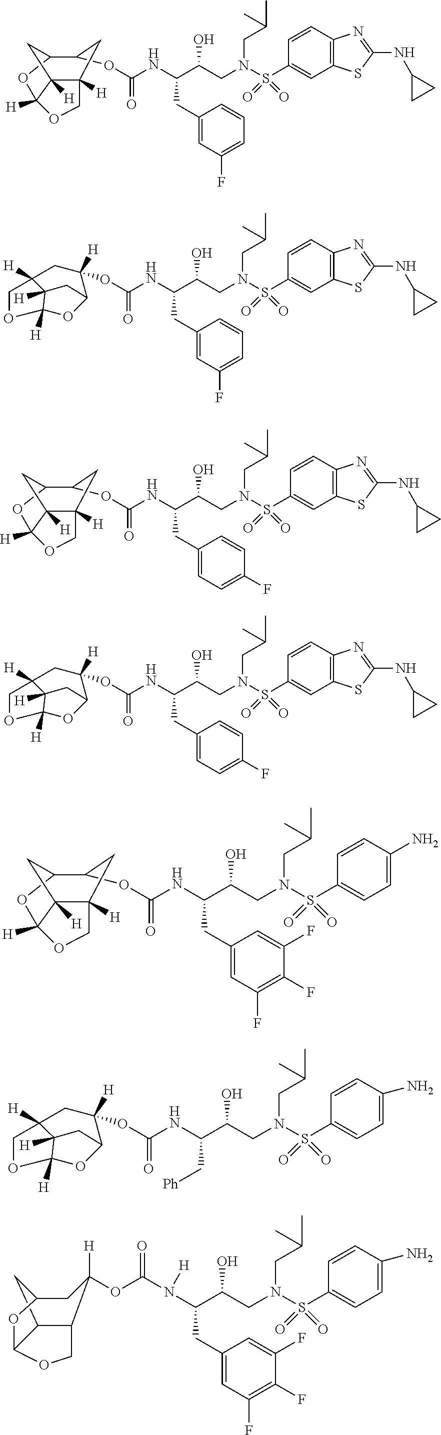

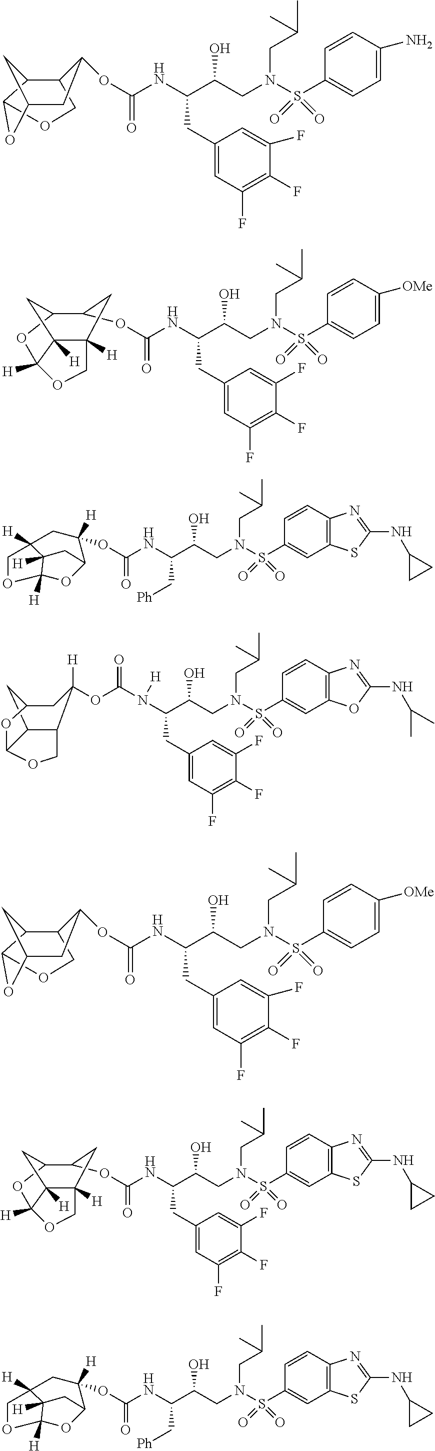

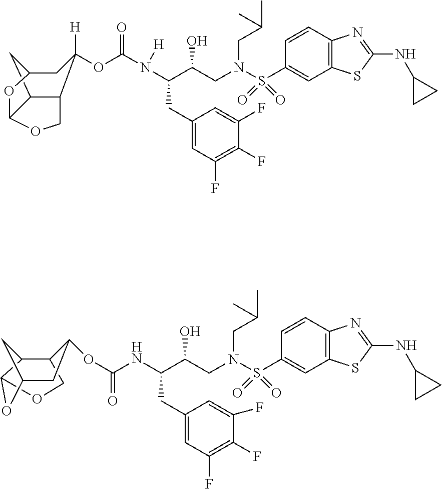 [SMILES: CC(C)CN(C[C@@H](O)[C@H](Cc1cc(F)c(F)c(F)c1)NC(=O)OC1CC2CC3C(OCC13)O2)S(=O)(=O)c1ccc2nc(NC3CC3)sc2c1 ; CC(C)CN(C[C@@H](O)[C@H](Cc1cc(F)c(F)c(F)c1)NC(=O)OC1CC2CC3C(OCC13)O2)S(=O)(=O)c1ccc2nc(NC3CC3)sc2c1]

or a pharmaceutically acceptable salt, polymorph, prodrug, solvate or clathrate thereof.

Embodiment 13 relates to a pharmaceutical composition comprising a compound of Embodiments 1-12 and one or more pharmaceutically acceptable excipients.

Embodiment 14 relates to a method for treating an HIV infection comprising administering a therapeutically effective amount of one or more compounds of Embodiments 1-12 to a patient in need thereof.

Embodiment 15 relates to a compound of Embodiment 1-12 for use as a medicament for treating a patient in need of relief from an HIV infection.

Embodiment 16 relates to a compound of formula (II)

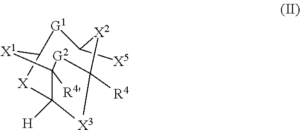

or a pharmaceutically acceptable salt, polymorph, prodrug, solvate or clathrate thereof, wherein:
n is an integer from 0 to 3; G¹ and G² are each independently (—CHR⁵—)ₚ, wherein p is 0 or 1 and each R⁵ is independently H or alkyl;
X is (—CHR⁵—)ₘO—, wherein m is 0, 1 or 2 and each R⁵ is independently H or alkyl;
X³ is (—CHR⁵—)_{d}O—, wherein d is 1 or 2 and each R⁵ is independently H or alkyl;
X¹ and X² are each, independently, (—CHR⁵—)ₘ, wherein m is 0, 1 or 2 and each R⁵ is independently H or alkyl; each R¹ is independently alkyl, alkoxy, aryl, heterocyclyl, halo, hydroxy or amino;
R⁴ and R^{4′} are each independently H or alkyl; and
X⁵ is selected from the group consisting of hydroxy, alkoxy, amino, C(O)R, C(O)OR, OC(O)OR, C(O)N(R)₂, OC(O)N(R)₂, C(S)N(R)₂, (CH₂)₀₋₂O(R)C(O)R, (CH₂)₀₋₂N(R)C(O)R, (CH₂)₀₋₂O(R)C(O)OR, (CH₂)₀₋₂O(R)C(O)OR or (CH₂)₀₋₂N(R)N(R)₂, wherein each R can be, independently, hydrogen, alkyl, acyl, cycloalkyl, aryl, aralkyl, heterocyclyl, heteroaryl, or heteroarylalkyl, wherein any alkyl, acyl, cycloalkyl, aryl, aralkyl, heterocyclyl, heteroaryl, or heteroarylalkyl or two R groups bonded to a nitrogen atom or to adjacent nitrogen atoms can together with the nitrogen atom or atoms form a heterocyclyl.

Embodiment 17 relates to the compound of Embodiment 16, wherein:

X¹ is (—CHR⁵—)ₘ, with m being 2 and X² being (—CHR⁵—)ₘ, with m being 1;
X¹ and X² are each (—CHR⁵—)ₘ, with each m being 1;
X¹ is (—CHR⁵—)ₘ, with m being 0 and X² is (—CHR⁵—)ₘ with m being 1; or
X¹ and X² are each (—CHR⁵—)ₘ, with m being 0, provided that at least one of G¹ and G² is (—CHR⁵—)ₚ, wherein at least one p is 1.

Embodiment 18 relates to the compound of Embodiment 16 or 17, wherein:

### X is O;

### X³ is O; or

### X and X³ are O.

Embodiment 19 relates to the compound of Embodiments 16-18, wherein at least one p is 0, such that at least one of G¹ and G² are a bond.

Embodiment 20 relates to the compound of Embodiments 16-19, wherein R⁴ and R^{4′} are each independently H or alkyl.

Embodiment 21 relates to the compound of Embodiments 16-18, wherein:

each p is 0;
X and X³ are each O,
X¹ and X² are each independently (—CHR⁵—)ₘ; and
R⁴ and R^{4′} are each H.

Embodiment 22 relates the compound of Embodiment 16, wherein the compound of formula (II) is a compound of formula:

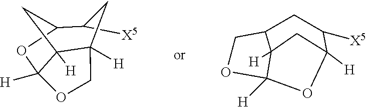

or a pharmaceutically acceptable salt, polymorph, prodrug, solvate or clathrate thereof.

What is claimed is:

1. A compound of the formula (I) 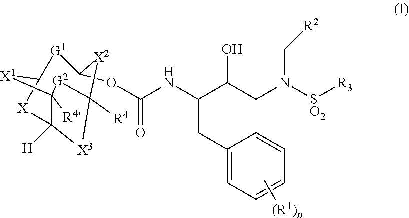
or a pharmaceutically acceptable salt, polymorph, prodrug, solvate or clathrate thereof, wherein:
n is an integer from 0 to 3; G¹ and G² are each independently (—CHR⁵—)ₚ;
p is 0 or 1;
X is (—CHR⁵—)ₘO—,
m is 0, 1 or 2;
X³ is (—CHR⁵—)_{d}O—;
d is 1 or 2;
X¹ and X² are each, independently, (—CHR⁵—)ₘ;
m is 0, 1 or 2;
each R¹ is independently alkyl, alkoxy, aryl, heterocyclyl, halo, hydroxy or amino;
R² is alkyl;
R³ is aryl, benzthiazole, benzoxazole, benzofuranyl or indolyl;
R⁴ and R^{4′} are each independently H or alkyl; and
and each R⁵ is independently H or alkyl.

2. The compound of claim 1, wherein
X¹ is (—CHR⁵—)ₘ, with m being 2 and X² being (—CHR⁵—)ₘ, with m being 1;
X¹ and X² are each (—CHR⁵—)ₘ, with each m being 1;
X¹ is (—CHR⁵—)ₘ, with m being 0 and X² is (—CHR⁵—)ₘ with m being 1; or
X¹ and X² are each (—CHR⁵—)ₘ, with m being 0, provided that at least one of G¹ and G² is (—CHR⁵—)ₚ, wherein at least one p is 1.

3. The compound of claim 1, wherein:
X is O;
X³ is O; or
X and X³ are O.

4. The compound of claim 1, wherein at least one p is 0, such that at least one of G¹ and G² are a bond.

5. The compound of claim 1, wherein R⁴ and R^{4′} are each independently H or alkyl.

6. The compound of claim 1, wherein:
each p is 0;
X and X³ are each O,
X¹ and X² are each independently (—CHR⁵—)ₘ; and
R⁴ and R^{4′} are each H.

7. The compound of claim 1, wherein the compound of formula (I) is a compound of formula:

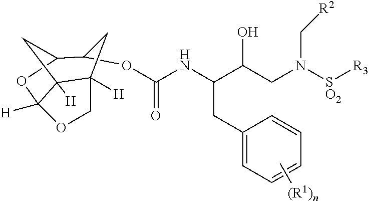

8. The compound of claim 1, wherein the compound of formula (I) is a compound of formula:

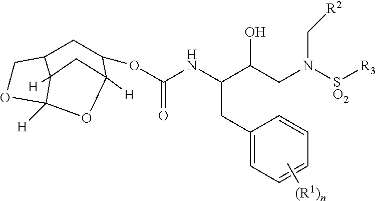

9. The compound of claim 1, wherein R³ is unsubstituted or substituted aryl.

10. The compound of claim 1, wherein R³ is selected from the group consisting of:

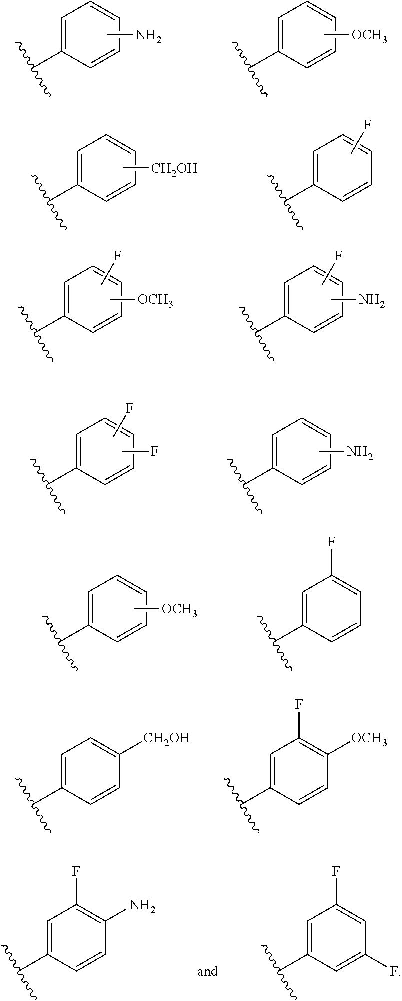

11. The compound of claim 1, wherein R³ is a benzthiazole or a benzoxazole: 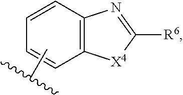
wherein R⁶ is alkyl, alkylamino, cycloalkylamino, cycloalkyl heterocycloamino, heterocyclo cycloalkylamino or heterocycloamino; and
X⁴ is S, O or NR⁷, wherein R⁷ is H, alklyl, cycloalkyl or alkylaryl. X⁴ can be S or O.

12. The compound of claim 1, wherein the compound is a compound of formula: 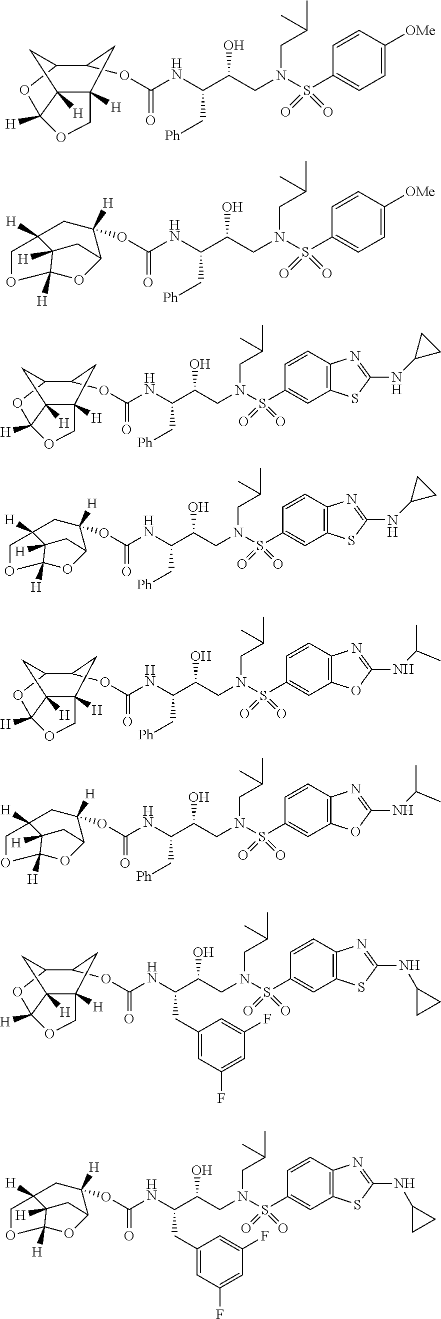 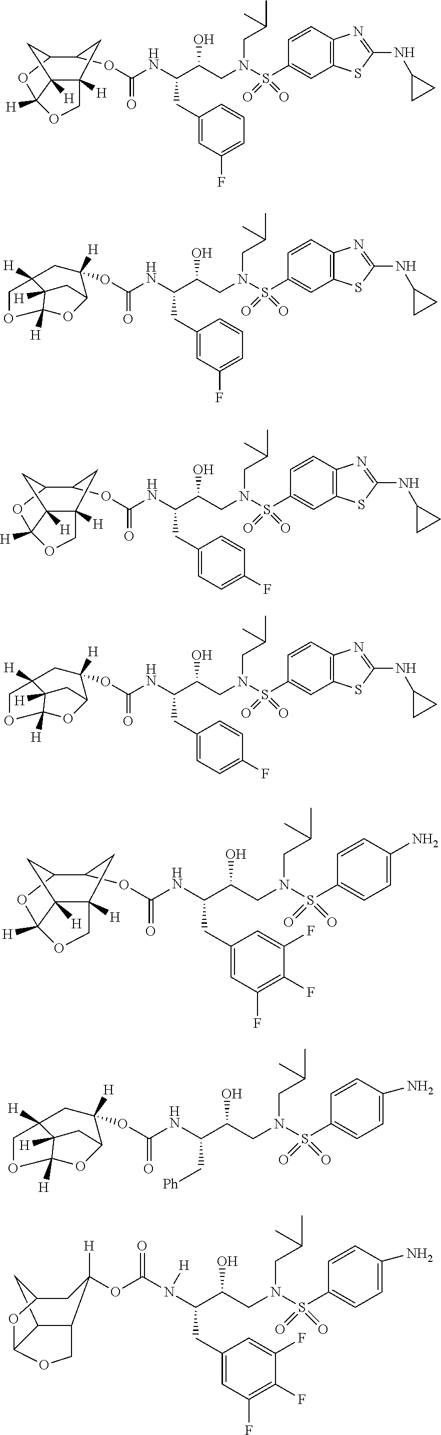 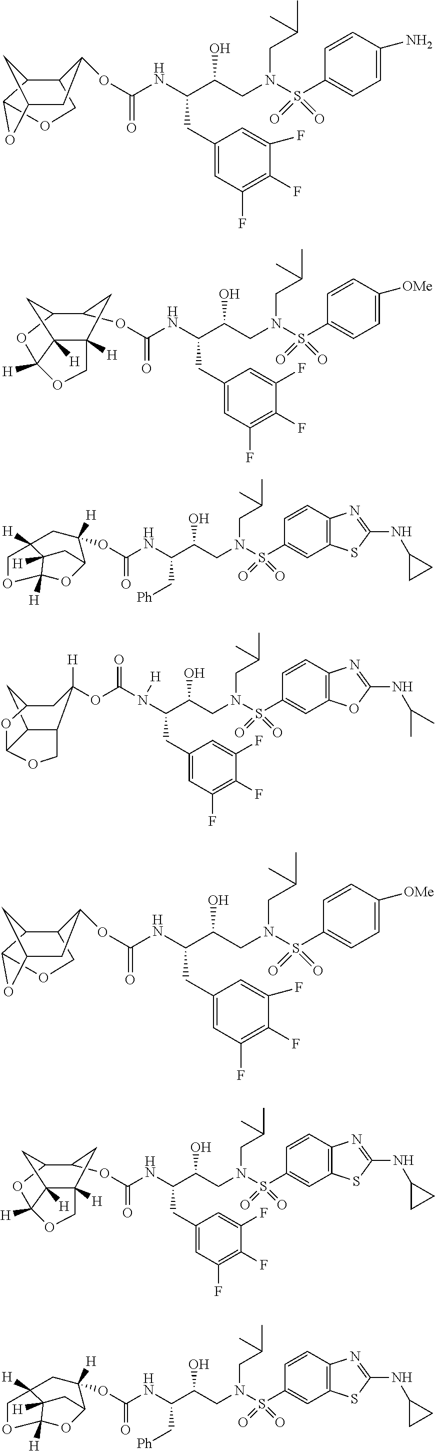 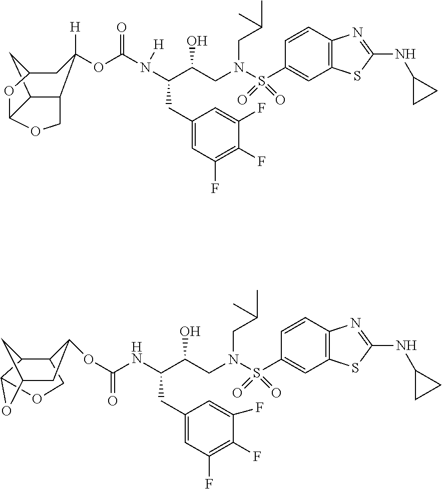 [SMILES: CC(C)CN(C[C@@H](O)[C@H](Cc1cc(F)c(F)c(F)c1)NC(=O)OC1CC2CC3C(OCC13)O2)S(=O)(=O)c1ccc2nc(NC3CC3)sc2c1 ; CC(C)CN(C[C@@H](O)[C@H](Cc1cc(F)c(F)c(F)c1)NC(=O)OC1CC2CC3C(OCC13)O2)S(=O)(=O)c1ccc2nc(NC3CC3)sc2c1]
or a pharmaceutically acceptable salt, polymorph, prodrug, solvate or clathrate thereof.

13. A pharmaceutical composition comprising a compound of claim 1 and one or more pharmaceutically acceptable excipients.

14. A method for treating an HIV infection comprising administering an effective amount of one or more compounds of claim 1 to a patient in need thereof.

15. A compound of formula (II) 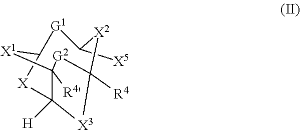
or a pharmaceutically acceptable salt, polymorph, prodrug, solvate or clathrate thereof, wherein:
n is an integer from 0 to 3; G¹ and G² are each independently (—CHR⁵—)ₚ, wherein p is 0 or 1 and each R⁵ is independently H or alkyl;
X is (—CHR⁵—)ₘO—, wherein m is 0, 1 or 2 and each R⁵ is independently H or alkyl;
X³ is (—CHR⁵—)_{d}O—, wherein d is 1 or 2 and each R⁵ is independently H or alkyl;
X¹ and X² are each, independently, (—CHR⁵—)ₘ, wherein m is 0, 1 or 2 and each R⁵ is independently H or alkyl; each R¹ is independently alkyl, alkoxy, aryl, heterocyclyl, halo, hydroxy or amino;
R⁴ and R^{4′} are each independently H or alkyl; and
X⁵ is selected from the group consisting of hydroxy, alkoxy, amino, C(O)R, C(O)OR, OC(O)OR, C(O)N(R)₂, OC(O)N(R)₂, C(S)N(R)₂, (CH₂)₀₋₂O(R)C(O)R, (CH₂)₀₋₂N(R)C(O)R, (CH₂)₀₋₂O(R)C(O)OR, (CH₂)₀₋₂O(R)C(O)OR or (CH₂)₀₋₂N(R)N(R)₂, wherein each R can be, independently, hydrogen, alkyl, acyl, cycloalkyl, aryl, aralkyl, heterocyclyl, heteroaryl, or heteroarylalkyl, wherein any alkyl, acyl, cycloalkyl, aryl, aralkyl, heterocyclyl, heteroaryl, or heteroarylalkyl or two R groups bonded to a nitrogen atom or to adjacent nitrogen atoms can together with the nitrogen atom or atoms form a heterocyclyl.

16. The compound of claim 15, wherein
X¹ is (—CHR⁵—)ₘ, with m being 2 and X² being (—CHR⁵—)ₘ, with m being 1;
X¹ and X² are each (—CHR⁵—)ₘ, with each m being 1;
X¹ is (—CHR⁵—)ₘ, with m being 0 and X² is (—CHR⁵—)ₘ with m being 1; or
X¹ and X² are each (—CHR⁵—)ₘ, with m being 0, provided that at least one of G¹ and G² is (—CHR⁵—)ₚ, wherein at least one p is 1.

17. The compound of claim 15, wherein:
X is O;
X³ is O; or
X and X³ are 0.

18. The compound of claim 15, wherein at least one p is 0, such that at least one of G¹ and G² are a bond.

19. The compound of claim 16, wherein R⁴ and R^{4′} are each independently H or alkyl.

20. The compound of claim 15, wherein:
each p is 0;
X and X³ are each O,
X¹ and X² are each independently (—CHR⁵—)ₘ; and
R⁴ and R^{4′} are each H.

21. The compound of claim 15, wherein the compound of formula (II) is a compound of formula: 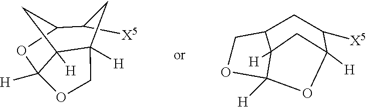
or a pharmaceutically acceptable salt, polymorph, prodrug, solvate or clathrate thereof.
